SEQUENCE LISTING (17 sequences)

SEQ ID NO: 1 - DNA, 1494 bp, Streptomyces eurythermus
  source 1..1494: mol_type = unassigned DNA
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaagaggcttcgctcatgcacatggacatcgacatcgaccaggacgtctgtatcggcgccgggcagtgcgcgctggcggcaccgggcgtcttcatccaggacgacgacggctacagcaccctgctgccaggccaggagaacggcgtcaccgacccgatggcccgggaggccgcccgcgcctgcccggtcagcgccatcaccgtacgggagcgctccgcctga

SEQ ID NO: 2 - DNA, 1494 bp, synthetic construct
  misc_feature 1..1494: note = Cloned version of SEQ ID NO: 1 with alternative stop codons
  source 1..1494: mol_type = other DNA
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaagaggcttcgctcatgcacatggacatcgacatcgaccaggacgtctgtatcggcgccgggcagtgcgcgctggcggcaccgggcgtcttcatccaggacgacgacggctacagcaccctgctgccaggccaggagaacggcgtcaccgacccgatggcccgggaggccgcccgcgcctgcccggtcagcgccatcaccgtacgggagcgctccgcctaa

SEQ ID NO: 3 - protein, 424 aa, Streptomyces eurythermus
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 4 - protein, 68 aa, Streptomyces eurythermus
MHMDIDIDQDVCIGAGQCALAAPGVFIQDDDGYSTLLPGQENGVTDPMAREAARACPVSAITVRERSA

SEQ ID NO: 5 - DNA, 56 bp, synthetic construct
  misc_feature 1..56: note = Primer Sequence
  source 1..56: mol_type = other DNA
attttgtttaactttaagaaggagatatacatatgaagatcggcacgacgcacctc

SEQ ID NO: 6 - DNA, 60 bp, synthetic construct
  misc_feature 1..60: note = Primer Sequence
  source 1..60: mol_type = other DNA
ctacccgcagagggcggggcataagcttcctattaggcggagcgctcccgtacggtgatg

SEQ ID NO: 7 - DNA, 53 bp, synthetic construct
  misc_feature 1..53: note = Primer Sequence
  source 1..53: mol_type = other DNA
gggtcctcaacgacaggagcacgatcatgccggaaatacaggaacgcacgctg

SEQ ID NO: 8 - DNA, 56 bp, synthetic construct
  misc_feature 1..56: note = Primer Sequence
  source 1..56: mol_type = other DNA
ttatcgccggcatggcggccccacgggtgccggggcacaactcaatttgcgggtac

SEQ ID NO: 9 - DNA, 1275 bp, Streptomyces eurythermus
  source 1..1275: mol_type = unassigned DNA
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 10 - DNA, 207 bp, Streptomyces eurythermus
  source 1..207: mol_type = unassigned DNA
atgcacatggacatcgacatcgaccaggacgtctgtatcggcgccgggcagtgcgcgctggcggcaccgggcgtcttcatccaggacgacgacggctacagcaccctgctgccaggccaggagaacggcgtcaccgacccgatggcccgggaggccgcccgcgcctgcccggtcagcgccatcaccgtacgggagcgctccgcctga

SEQ ID NO: 11 - protein, 454 aa, Streptomyces griseus
MPRPLRVAIVGSGPAGIYAADALLKSEVAADPGVSIDIFERMPAPFGLIRYGVAPDHPRIKGIITALHQVLDKPQIRLFGNVNYPTDVSLDDLRAFYDGVIFATGATADRDLSLPGIDLDGSYGAADFVAWYDGHPDFPRTWPLEAEKVAVLGVGNVALDIARVLAKTADELLPTEIPPNVYEGLKANKALEVHVFGRRGPAQAKFSPMELRELDHSPNIEVIVDPEDIDYDEGSIATRRGNKQADMVAKTLENWAIRDVGDRPHKLFLHFFESPAEILGEDGRVTGLRTERTELDGTGNVKGTGEFKDWDVQAVYRAVGYLSDQLPKLPWDLETGTVPDAGGRVVQESGEHLQSTYVTGWIRRGPIGLIGHTKGDANETVSNLLDDYANGRLQTPSSPAPEAVDAFLAERNVRFTTWDGWYRLDAAEKAQGEPHGRERVKYVEREDMLRESGA

SEQ ID NO: 12 - DNA, 1365 bp, Streptomyces griseus
  source 1..1365: mol_type = unassigned DNA
atgccccgccctctgcgggtagccatcgtcggatccggcccggccgggatctacgccgccgacgccctgctcaagtccgaagtggccgccgaccccggtgtttccatcgacatcttcgagcgcatgcccgccccgttcggcctcatccggtacggcgtcgcgcccgaccacccgcggatcaagggcatcatcacggccctccaccaggtgctcgacaagccgcagatccgcctcttcggcaacgtgaactaccccaccgacgtcagcctggacgatctgcgcgccttctacgacggtgtgatcttcgccaccggcgccacggcggaccgggacctgtccctcccgggcatcgacctcgacggctcgtacggcgcggccgacttcgtcgcctggtacgacggccaccccgacttcccgcgcacctggccgctggaggcggagaaagtcgccgtcctcggtgtcggcaacgtcgccctggacatcgcgcgcgtcctcgccaagacggccgacgagctgctgccgaccgagatcccgccgaacgtctacgagggcctcaaggccaacaaggcgctggaggtgcacgtcttcggccgccgcggcccggcgcaggcgaagttcagcccgatggagctgcgggagctggaccactcccccaacatcgaggtgatcgtcgaccccgaggacatcgactacgacgagggctcgatcgcgacccggcgcggcaacaagcaggccgacatggtcgccaagaccctggagaactgggcgatccgcgacgtcggcgaccggccgcacaagctgttcctgcacttcttcgagtcgcccgcggagatcctcggcgaggacggcagggtgaccggcctgcgcaccgagcgcacggagctggacggcacgggcaacgtcaagggcaccggcgagttcaaggactgggacgtccaggcggtctaccgggccgtcggctacctctccgaccagctgcccaagctgccctgggacctcgagacgggcacggtcccggacgcgggcggccgggtcgtccaggagtccggcgagcacctccagtcgacgtacgtcaccggctggatccggcgcggtccgatcggcctgatcggccacaccaagggcgacgccaacgagacggtgtccaacctgctggacgactacgcgaacggccgtctccagacgccctcctcccccgctcccgaggccgtggacgcgttcctcgccgagcggaacgtccgcttcaccacctgggacggctggtaccggctcgacgccgcggagaaggcgcagggcgaaccgcacgggcgtgagcgcgtgaagtacgtcgagcgcgaggacatgctccgcgagagcggcgcctaa

SEQ ID NO: 13 - protein, 404 aa, Amycolatopsis lurida
MTDVEETTATLPLARKCPFSPPPEYERLRRESPVSRVGLPSGQTAWALTRLEDIREMLSSPHFSSDRQSPSFPLMVARQIRREDKPFRPSLIAMDPPEHSRARRDVVGEFTVKRMKALQPRIQQIVDEHLDALLAGPKPADLVQALSLPVPSLVICELLGVPYSDHEFFQSCSSRMLSREVTAEERMTAFEQLENYLDELVTKKEANATEDDLLGRQILKQRETGEADHGELVGLAFLLLIAGHETTANMISLGTVTLLENPDQLAKIKADPGKTLAAIEELLRVFTIAETATSRFATADVEIGGTLIRAGEGVVGLSNAGNHDPDGFENPDTFDIERGARHHVAFGFGVHQCLGQNLARLELQIVFDTLFRRVPGLRIAVPVDELPFKHDSTIYGLHALPVTW

SEQ ID NO: 14 - protein, 395 aa, Streptomyces rimosus
MTEALPFPQDRTCPYDPPAGYQPLRDSRPLSRVTLYDGRPAWVVTGHAESRALLTDPRLSADRQNPAFPSPAPRFETLRKVRTPLLGVDDPEHNTQRRMLIPSFSVKRAAALRPRIQEIVDRLLDAMEQQGPPAELVSAFALPVPSMVICALLGVPYADHELFEGLSRTLLQSADPQEVTEARDKLEDYFTALVERKRKEPGDGLLDELIAERLDSGELGHRELVRMAMLLLVAGHETTSNMLSLGTFTLLEHPEQFAALRADPSLLPAAVEELLRFLSIADGMVRVATEDIEIGGVTIRADDGVIFSTSVVNRDGAAYASPDTLDWERSARHHVAFGFGVHQCLGQNLARAEMEIAFGALFARFPGLRLAVPAAEIPVKPAHALQGLVELPVTW

SEQ ID NO: 15 - protein, 403 aa, Streptomyces rimosus
MPEIIDLGAYGPDFVADPHPYYAKLRAQGPVHRVRAPEMEPEFPQAWLIVGYDEARAVLADNRFAKDWSRANGSLADSEVLAEWQLMNMLDADPPQHTRLRKLVAREFTTRRVEALRPRVQQITDELLDAMLAAPDGRADLVEALAFPLPMTVICELLGVPDIERDTFRAWSNELVSPTDNEATMTAAREMAAYLDGLIESKRSSPGEDLLSALVRTSDEDGDQLSRQELLGMAFLLLVAGHETTVNLISNGVRALLQHPAQLAALRADPSLLDNAVEEMLRYDGPVETATWRFTAEPVGIGGVEIPAGEIVLVGLAGADRDPERFEAPDTFDITRETRGHVAFGHGIHFCLGAPLARVEGRIAVRTLLDRCPDLALDTAPEALTWRAGMTIRGPQHLPVRWR

SEQ ID NO: 16 - protein, 454 aa, Streptomyces rimosus
MTTSPTESTTATPPDSTTASAPGTPPDALPSYVGLHPGEPNVMEPELLNDPYAGYGKLREQGALVRGRFLDDSPVWLVTRFDVVREVMRDPRFINNPSRLPGRTEKDPRAQLIELFGIPDHMARYLVDTILTSDPPDHTRLRRLVSRAFTARRIQDLRPRVEAITDELLDRLPAHAQDGVVDLVEHFAYPLPITVICELVGIDEEDRPLWRQFGADLTSLEPKRIGATVPAMVEHIHKVIGERQSALRDDLLSALIRARDDDGGRLSETEMVTMVLTLVLAGHETTAHLISNGTLALLTHPDQRRLLTEDPGLLPRAVHELMRWCGPIQATQLRYASEDVEVAGTQVHKGDALMFSLVAANHDPRHYTEPEKLDLTRQPAGRAEDHVGFGHGMHYCLGASLARQEGEVAFGKLLARYPEVALALPHEQLEEQERIRQPGSWRLRRLPLRLRPED

SEQ ID NO: 17 - protein, 399 aa, Streptomyces platensis
MAEIVDLGAYGKDFTRDPYPYYAKLRAQGPIHRVRLPYGNDVWLVVGHQAVRAALSDSRLNKDWRAAMPQGDVGEESALFTNMLDADPPQHTRLRKLVAKEFTSRRVEALRPRVQQITDELLDTMLAAPDGRADLVEALAFPLPMTVICELLGVPSMDRDAFRSWSNEIVAPTSDEAAQAAVVAMSGYLVELIETKRNAPGDGLLSALIRTSDEDGDQLSRDELVGTAFLLLVAGHETTVSLLANGVRALLQHPDQLAALRADFSLLDNAVEEMLRYDGPVETATWRFTGEPVEIGGTLIPAGETVAIALASASRDPEIFAVADDFDITRDARGHTAFGHGIHFCLGAPLARLEARIALRSLLERCPDLAMDADFDDLTWRVGMLRGPARLPVRWKSWS

(19) United States
(12) Patent Application Publication
(10) Pub. No.: US 2025/0388875 A1
(43) Pub. Date: Dec. 25, 2025

(54) BIOCATALYTIC TECHNIQUES

(71) Applicant: HYPHA DISCOVERY LIMITED, Abingdon Oxfordshire (GB)

(72) Inventors: Jonathan Charles Paul STEELE, Abingdon Oxfordshire (GB); Antonio DE RISO, Abingdon Oxfordshire (GB); Francesco FALCIONI, Manchester Greater Manchester (GB); Stephen Keith WRIGLEY, Abingdon Oxfordshire (GB); Emily Jade HOPKINS, Abingdon Oxfordshire (GB); Aksana Rimu KHAN, Abingdon Oxfordshire (GB); Kinga Linda NYTKO, Abingdon Oxfordshire (GB); Vincent POON, Abingdon Oxfordshire (GB); John Maxim WARD, London (GB); Sebastian SCHULZ, Schenkendöbern (DE)

(73) Assignee: HYPHA DISCOVERY LIMITED, Abingdon Oxfordshire (GB)

(21) Appl. No.: 19/303,698

(22) Filed: Aug. 19, 2025

Related U.S. Application Data
(62) Division of application No. 17/778,315, filed on May 19, 2022, now Pat. No. 12,410,411.

(30) Foreign Application Priority Data
Nov. 22, 2019 (GB) 1917077.8

(51) Int. Cl. C12N 9/02; C12N 15/70; C12P 7/26; C12P 7/6409; C12P 13/00; C12P 13/02; C12P 17/04; C12P 17/10; C12P 17/12; C12P 17/16; C12P 17/18

(52) U.S. Cl. CPC C12N 9/0071; C12N 15/70; C12P 7/26; C12P 7/6409; C12P 13/001; C12P 13/02; C12P 17/04; C12P 17/10; C12P 17/12; C12P 17/16; C12P 17/165; C12P 17/167; C12P 17/181; C12P 17/182; C12Y 114/14001

(57) ABSTRACT

The present invention relates to a cytochrome P450 enzyme comprising the amino acid sequence set forth in SEQ ID NO: 3, or a variant thereof having an amino acid sequence having at least 95% identity thereto and having CYP450 activity. The cytochrome P450 enzyme provided herein was isolated from Streptomyces eurythermus NRRL 2539 and has a wide substrate range and high activity, and may be used to oxidate organic compounds.

### SEQUENCE LISTING

A sequence listing in electronic (XML file) format is filed with this application and incorporated herein by reference. The name of the XML file is “Sequence_Listing-1165.xml”; the file was created on Aug. 11, 2025; the size of the file is 26,529 bytes.

### FIELD OF THE INVENTION

The present invention relates to a cytochrome P450 enzyme from Streptomyces eurythermus NRRL 2539, nucleic acids encoding the enzyme, kits comprising the enzyme, and uses of the enzyme for catalysing the oxidation of organic substrates.

### BACKGROUND OF INVENTION

Cytochrome P450 (CYP) is a superfamily of haem-thiolate proteins named for the spectral absorbance peak of their carbon-monoxide bound species at 450 nm. They are found in all kingdoms of life such as animals, plants, fungi, protists, bacteria, archaea, and furthermore a putative P450 from giant virus Acanthamoeba polyphaga has been proposed, Lamb, D C; Lei, L; Warrilow, A G; Lepesheva, G I; Mullins, J G; Waterman, M R; Kelly, S L (2009). “The first virally encoded cytochrome P450”. Journal of Virology. 83 (16): pp 8266-9. Cytochrome P450 enzymes have not been identified in E. coli, Roland Sigel; Sigel, Astrid; Sigel, Helmut (2007). The Ubiquitous Roles of Cytochrome P450 Proteins: Metal Ions in Life Sciences. New York: Wiley. ISBN 0-470-01672-8; Danielson P B (December 2002). “The cytochrome P450 superfamily: biochemistry, evolution and drug metabolism in humans”. Curr. Drug Metab. 3 (6): pp 561-97.

Cytochrome P450s show extraordinary diversity in their reaction chemistry supporting the oxidative, peroxidative and reductive metabolism of a diverse range of endogenous and xenobiotic substrates.

In humans, cytochrome P450s are best known for their central role in phase I drug metabolism where they are of critical importance for two of the most significant problems in clinical pharmacology: drug-drug interactions and inter-individual variability in drug metabolism.

The most common reaction catalyzed by cytochromes P450 is a mono-oxygenase reaction. Cytochrome P450 mono-oxygenases use a haem group to oxidise molecules, often making them more water-soluble by either adding or unmasking a polar group. In general the reactions catalysed by these enzymes can be summarised as:

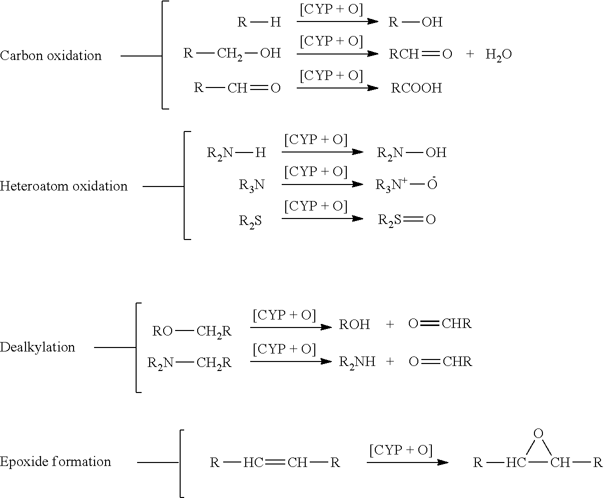

In the first line example, R—H is the substrate and R—OH is the oxygenated substrate. The oxygen is bound to the haem group in the core of the CYP enzyme, protons (H⁺) are usually indirectly derived from the reduced cofactor NADH or NADPH via redox partner proteins, either discrete proteins or fused to the CYP, through specific amino acids in the CYP enzyme. CYP enzymes can receive electrons from a range of redox partner proteins such as cytochrome b5, a ferredoxin reductase and a ferredoxin, and adrenodoxin reductase and adrenodoxin.

Although classification and nomenclature of cytochrome P450 is quite complex, they can be classified by their redox partner transfer protein system, proposed by I. Hanukoglu (1996). “Electron Transfer Proteins of Cytochrome P450 Systems”. Advances in Molecular and Cell Biology. Advances in Molecular and Cell Biology. 14: 29-56. In summary, cytochrome P450 enzymes can be classified into the following groups:
Microsomal P450 systems which utilise cytochrome P450 reductase or cytochrome b5 to transfer electrons from cofactor to cytochrome P450;
Mitochondrial P450 systems which utilise adrenodoxin reductase and adrenodoxin to transfer electrons from reduced cofactor to cytochrome P450;
Bacterial P450 systems which utilise ferredoxin reductase and ferredoxin proteins to transfer electrons from reduced cofactor to cytochrome P450;
CYBER-cytb5-P450 systems, which utilise cytochrome b5 for the electron transfer from the cofactor to the cytochrome P450;
FMN-Fd-P450 systems in which the electron partner reductase is a fused FMN domain;
P450 only systems that do not require redox partner proteins, e.g., P450_{BM-3}.

Isolated bacterial cytochrome P450 enzymes are known, including P450_{cam} from Pseudomonas putida, J Biol Chem (1974) 249, 94; P450_{BM-1} and P450_{BM-3} both from Bacillus megaterium ATCC 14581, Biochim Biophys Acta (1985) 838, 302, and J Biol Chem (1986) 261, 1986, 7160; P450a, P450b, and P450c from Rhizobium japonicum, Biochim Biophys Acta (1967) 147, 399; and P450npd from Nocardia NHI, Microbios (1974) 9, 119.

However, cytochrome P450 enzymes purified from Actinomycete microorganisms remain relatively unreported. The induction of a cytochrome P450 in Streptomyces griseus by soybean flour (P450_{soy}) is described in Biochem and Biophys Res Comm (1986) 141, 405. Other reported examples include the isolation and properties of two forms of a P450 effecting pesticide inactivation (P450_{SU1} & _{SU2}) and two forms of 6-deoxyerythronolide B hydroxylase from Saccharopolyspora erythraea (originally classified as Streptomyces erythraeus) as described in Biochemistry (1987) 26, 6204. U.S. Pat. No. 6,884,608 describes enzymatic hydroxylation of epothilone B to epothilone F, effected with a hydroxylation enzyme produced by a strain of Amycolatopsis orientalis (originally classified as Streptomyces orientalis). A more recent example is CYP107L from Streptomyces platensis DSM40041, reported in Biotechnology and Bioengineering (2018) 115; 2156-2166 for exhibiting activities resembling some human drug metabolising P450 enzymes.

In the field of medicinal chemistry, modifications to chemical compounds are used to alter the properties of such chemical compounds. For example, tertiary butyl moieties are often used by medicinal chemists in the synthesis of drug-like molecules for introduction of hydrophobicity. However, further modifications thereof can be used to improve potency, selectivity and solubility profiles of such compounds, for example hydroxylations can be used. Hydroxylations are also the main route of metabolic degradation, another important aspect of pharmacology and medicinal chemistry. Methods for the production of these hydroxylated metabolites are sought using biotransformation with animal tissues due to being often challenging to synthesise by purely chemical means.

### SUMMARY OF THE INVENTION

It has surprisingly been found that a specific cytochrome P450 enzyme found in Streptomyces eurythermus NRRL 2539 can be used for providing different types of oxidation reactions upon a range of organic substrates, the term oxidation and terms derived thereof referring to reaction types including but not limited to hydroxylation, epoxidation, carboxylation and dealkylation of the substrates.

In particular, cytochrome P450 enzyme having the amino acid sequence shown as SEQ ID NO: 3 can be used for the oxidation of organic compounds in order to activate or modify a compound's physicochemical and pharmacological properties. In a particularly preferred embodiment, the cytochrome P450 enzyme having the amino acid sequence shown as SEQ ID NO: 3 is useful for the oxidation of a variety of aliphatic and aromatic moieties, or chemicals containing such moieties, for the purposes of C—H activation or modification of a compound's physicochemical and pharmacological properties. The cytochrome P450 enzyme of SEQ ID NO: 3 has not previously been identified and due to its wide reactivity and superior activity on a number of substrates is a particularly useful cytochrome P450 for industrial use.

In a first aspect, the invention provides a cytochrome P450 enzyme comprising the amino acid sequence set forth in SEQ ID NO: 3, or a variant thereof having an amino acid sequence having at least 95% identity thereto and having CYP450 activity.

In a second aspect, the invention provides a nucleic acid molecule comprising a nucleotide sequence encoding an enzyme of the invention.

In a third aspect, the invention provides a recombinant construct comprising a nucleic acid molecule of the invention operatively linked to a heterologous expression control sequence.

In a fourth aspect, the invention provides a vector comprising the nucleic acid molecule or recombinant construct of the invention.

In a fifth aspect, the invention provides a microorganism comprising the nucleic acid molecule, the recombinant construct or the vector of the invention, wherein the nucleotide sequence encoding the enzyme of the invention is heterologous to the microorganism, wherein preferably the microorganism is not Streptomyces eurythermus.

In a sixth aspect, the invention provides the use of a cytochrome P450 enzyme comprising SEQ ID NO: 3 or a variant thereof having at least 95% identity thereto and having CYP450 activity, for the oxidation of an organic compound.

In a seventh aspect, the invention provides a method for the production of an oxidised organic compound, comprising reacting the organic compound with a cytochrome P450 enzyme comprising SEQ ID NO: 3 or a variant thereof having at least 95% identity thereto and having CYP450 activity.

In an eighth aspect, the invention provides a kit comprising:
i) a cytochrome P450 enzyme comprising SEQ ID NO: 3 or a variant thereof having at least 95% identity thereto and having CYP450 activity;
ii) a microorganism that expresses a cytochrome P450 enzyme comprising SEQ ID NO: 3 or a variant thereof having at least 95% identity thereto and having CYP450 activity, or a lysate of said microorganism; and/or
iii) a nucleic acid molecule, recombinant construct or vector of the invention; optionally wherein the kit further comprises instructions and other cofactor reagents for use for the oxidation of an organic compound.

In a ninth aspect, the invention provides a method of producing a cytochrome P450 enzyme of the invention, the method comprising introducing a nucleic acid molecule, a recombinant construct or a vector of the invention, into a microorganism, and expressing the cytochrome P450 enzyme in the microorganism, and optionally isolating and/or purifying the cytochrome P450 enzyme.

### BRIEF DESCRIPTION OF THE FIGURES

The invention is described with reference to the accompanying drawings, wherein:

FIG. 1 shows schematic examples of biotransformations effected by the use of the cytochrome P450 enzyme comprising SEQ ID NO: 3 of the present invention. FIG. 1(a) shows epoxidation of carbamazepine, FIG. 1(b) shows hydroxylation of bosentan (with demethylation as a minor side reaction), FIG. 1(c) shows hydroxylation of diclofenac, FIG. 1(d) shows hydroxylation of a methyl group of meloxicam, with some further oxidation to yield a carboxyl moiety, FIG. 1(e) shows hydroxylation of tivantinib, and FIG. 1(f) shows hydroxylation of ambroxide;

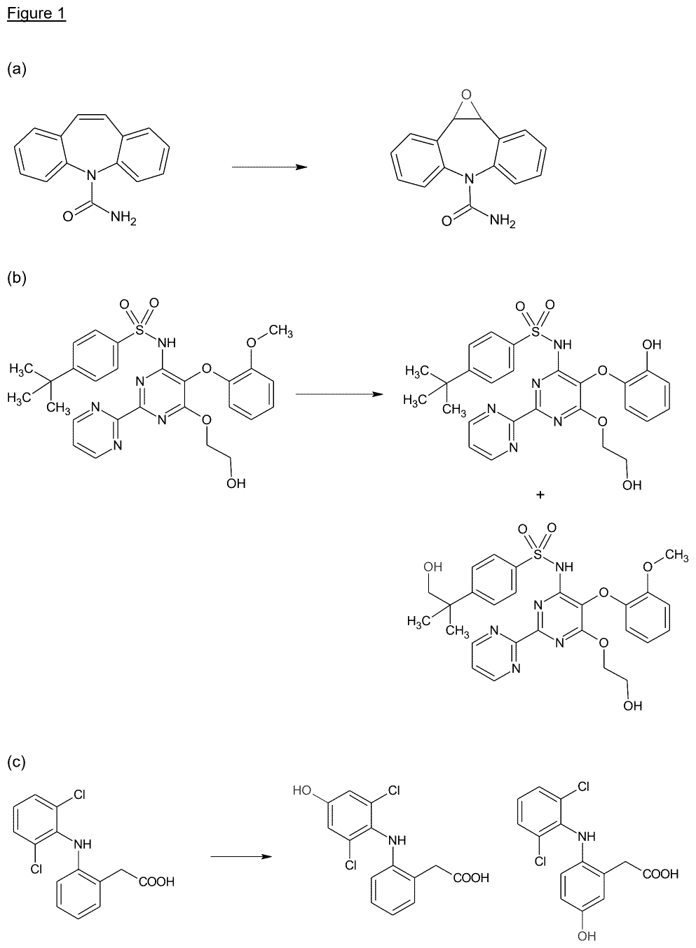

FIG. 2 shows expression plasmid pHD05-SeuC10-SeuF08.

FIG. 3 shows the carbon monoxide difference spectrum of the crude enzyme extract containing P450_{SeuC10} protein. The sample was prepared from IPTG-induced culture of E. coli Tuner (DE3) cells containing the pHD05-SeuC10-SeuF08 plasmid; and

FIGS. 4A-4F show UPLC-MS chromatograms of various reactions performed at the 100 μL screening scale:

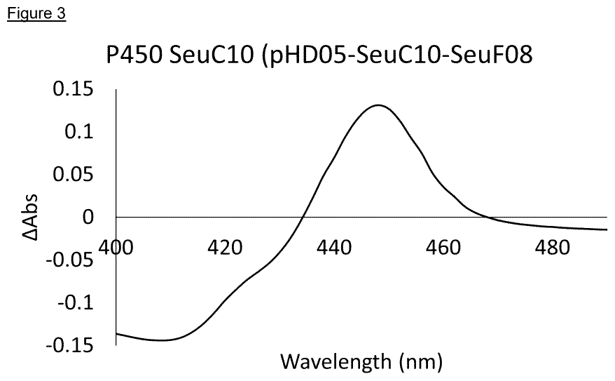

FIG. 4A shows chromatograms of post-reaction extract using lyophilised material of recombinant P450_{SeuC10}, ferredoxin_{seuF08} and ferredoxin reductase_{SCF15A}, as described in Example 4A, dosed with 100 mg/L carbamazepine. Top to bottom is UV₂₂₅ₙₘ, EIC_{237m/z} (carbamazepine, 1.33 mins) and EIC_{253m/z} (carbamazepine-10,11-epoxide, 1.16 mins (49% inferred yield));

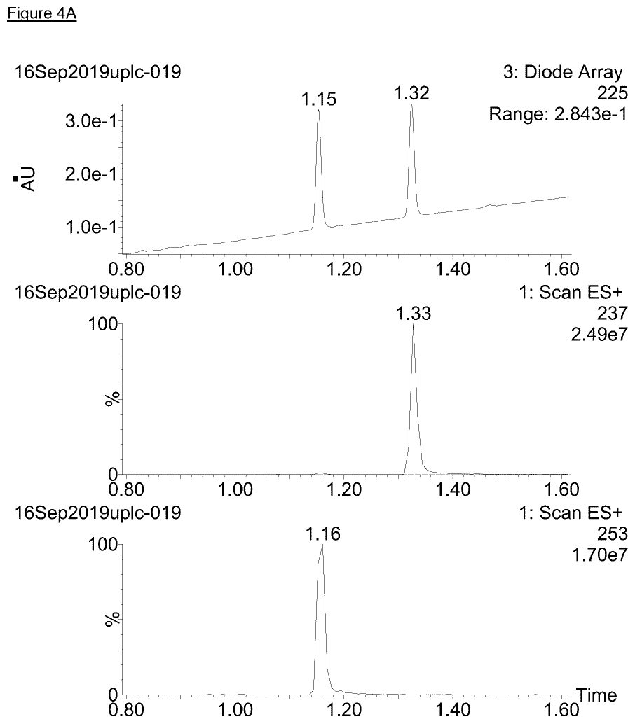

FIG. 4B shows chromatograms of post-reaction extract using E. coli expressing recombinant P450_{SeuC10}, ferredoxin_{seuF08} and ferredoxin reductase_{SCF15A}, as described in Example 4B, dosed with 100 mg/L bosentan. Top to bottom is UV₂₆₈ₙₘ, EIC_{552m/z} (bosentan, 1.76 mins)), EIC_{538m/z} (O-desmethylbosentan, 1.67 minute (3.4% inferred yield)) and EIC_{568m/z} (hydroxy-bosentan, 1.44 mins (81% inferred yield));

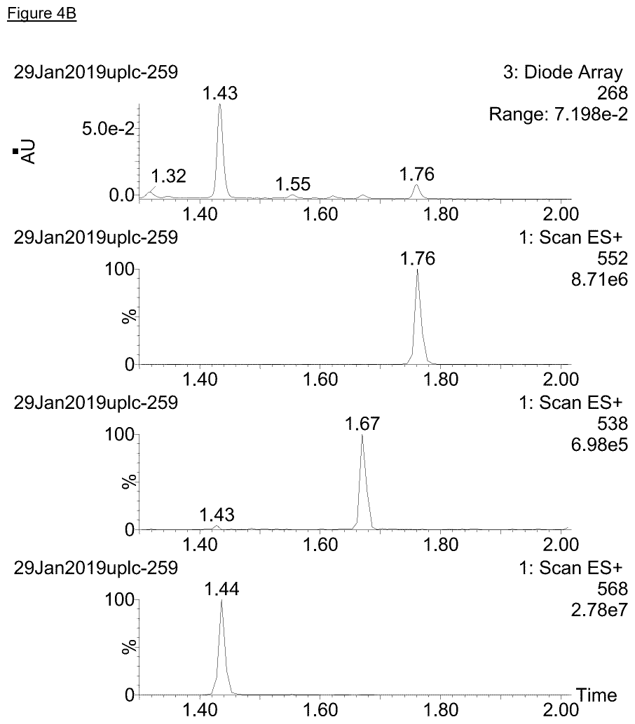

FIG. 4C shows chromatograms of post-reaction extract using E. coli expressing recombinant P450_{SeuC10}, ferredoxin_{seuF08} and ferredoxin reductase_{SCF15A}, as described in Example 4B, dosed with 100 mg/L diclofenac. Top to bottom is UV₂₇₅ₙₘ, EIC_{294m/z} (diclofenac, 1.83 mins)), EIC_{310m/z} (5-hydroxydiclofenac and 4′-hydroxydiclofenac, 1.59 minutes (19.4% inferred yield) and 1.64 minutes (73.4% inferred yield), respectively);

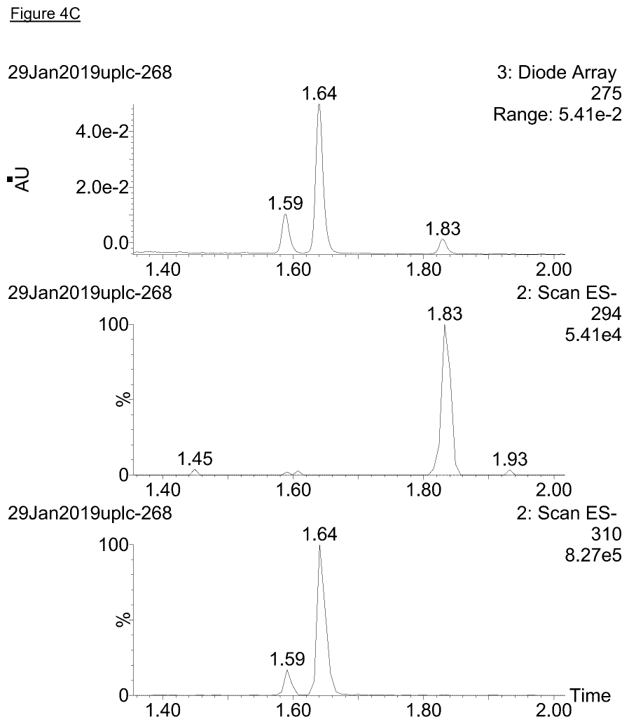

FIG. 4D shows chromatograms of post-reaction extract using lyophilised material of recombinant P450_{SeuC10}, ferredoxin_{seuF08} and ferredoxin reductase_{SCF15A}, as described in Example 4A, dosed with 100 mg/L meloxicam. Top to bottom is UV₃₅₄ₙₘ, EIC_{352m/z} (meloxicam, 1.61 mins), EIC_{368m/z} (5′-hydroxymethylmeloxicam, 1.32 mins (28.4% inferred yield) and EIC_{382m/z} (5′-carboxylmeloxicam, 1.54 mins (5.3% inferred yield));

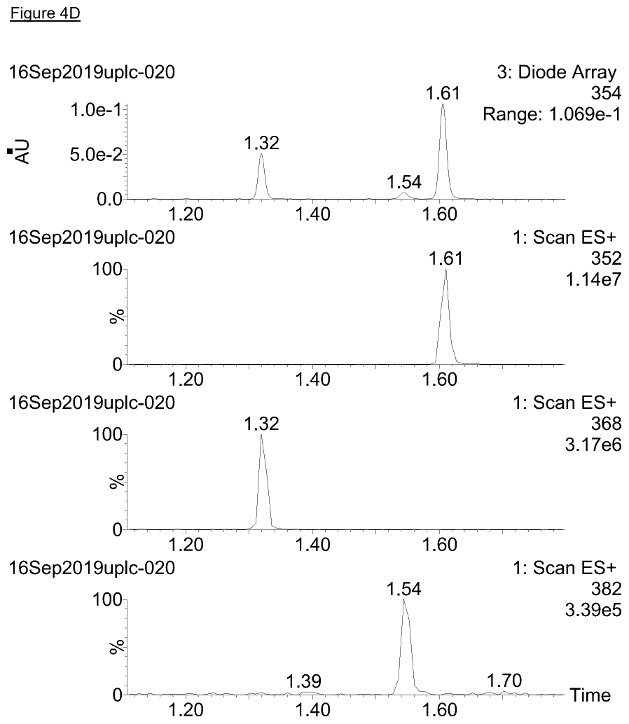

FIG. 4E shows chromatograms of post-reaction extract using lyophilised material of recombinant P450_{SeuC10}, ferredoxin_{seuF08} and ferredoxin reductase_{SCF15A}, as described in Example 4A, dosed with 100 mg/L tivantinib. Top to bottom is UV₂₈₀ₙₘ, EIC_{370m/z} (tivantinib, 1.55 mins) and EIC_{386m/z} (epimeric benzylic hydroxylation products of tivantinib, 1.16 and 1.14 mins (98.3% combined inferred yield));

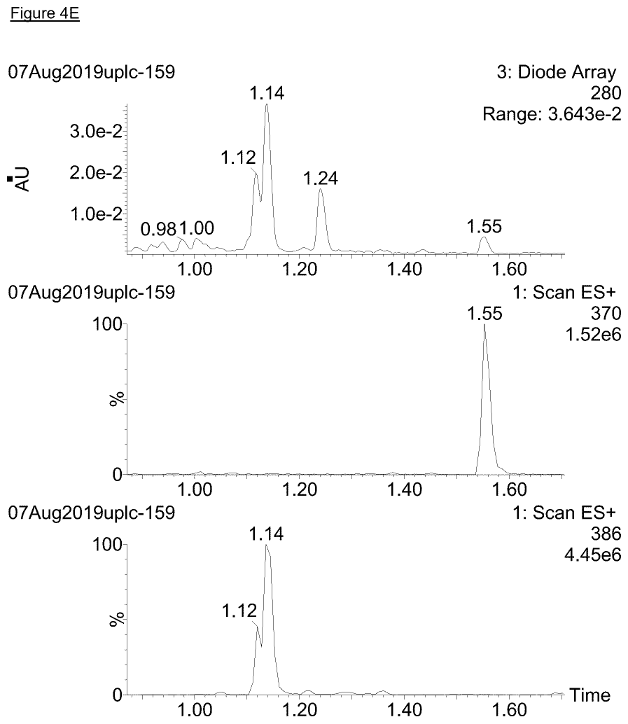

FIG. 4F shows chromatograms of post-reaction extract using lyophilised material of recombinant P450_{SeuC10}, ferredoxin_{seuF08} and ferredoxin reductase_{SCF15A}, as described in Example 4A, dosed with 100 mg/L ambroxide. Top to bottom is EIC_{237m/z} (ambroxide, 2.19 mins) and EIC_{253m/z} (hydroxy-ambroxide derivative, 1.46 mins (74% inferred yield)).

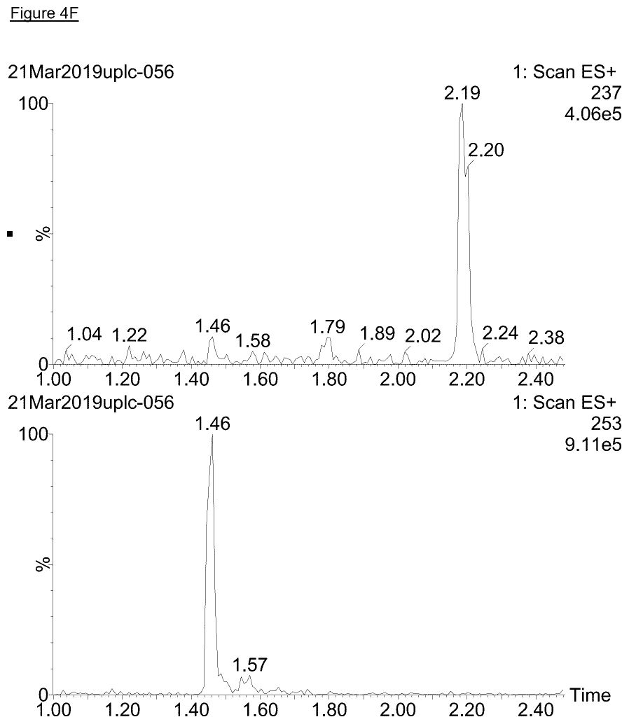

### DESCRIPTION OF THE PREFERRED EMBODIMENTS

The first aspect of the invention provides a cytochrome P450 enzyme comprising the amino acid sequence set forth in SEQ ID NO: 3, or a variant thereof having an amino acid sequence having at least 95% identity thereto and having CYP450 activity. This aspect of the invention may alternatively be seen as providing a polypeptide having cytochrome P450 activity, and comprising the amino acid sequence set forth in SEQ ID NO: 3 or a sequence with at least 95% identity thereto. In preferred embodiments, the enzyme comprises an amino acid sequence having at least 96%, 97%, 98% or 99% identity to SEQ ID NO: 3. Most preferably the enzyme comprises the amino acid sequence of SEQ ID NO: 3. In some embodiments, the enzyme may consist of the amino acid sequence of SEQ ID NO: 3, or an amino acid sequence having at least 95, 96, 97, 98 or 99% identity thereto. The origin of the enzyme and methods by which it may be obtained are described below. Similarly, variants of the enzyme falling within the invention are described below, including preferred substitutions etc. by which a variant may be obtained.

The enzyme of the invention is isolated relative to its native or natural form. Thus the enzyme is separated from other components with which it is normally associated. For example, the enzyme may normally be present in a microorganism, but in this aspect of the invention, it is separated from at least some components of that microorganism. The isolated enzyme may take the form of an enriched extract in which the enzyme's concentration is increased relative to its concentration in the microorganism or a simple, untreated, extract thereof. In a preferred aspect, the isolated enzyme is the primary component (i.e. majority component) of any solution or suchlike in which it is provided. In particular, if the enzyme is initially produced in a mixture or mixed solution, the enzyme may be separated or purified therefrom. Thus, for instance, if the enzyme is produced using a protein expression system (such as a cellular expression system using prokaryotic (e.g. bacterial) cells, a cell-free, in vitro expression system), the enzyme may be isolated such that it is the most abundant polypeptide in the solution or composition in which it is present, preferably constituting the majority of polypeptides in the solution or composition, and is enriched relative to other polypeptides and biomolecules present in the native production medium. As discussed below, generally the enzyme of the invention is produced using a cellular expression system, in particular by expression in bacterial cells.

In a preferred feature, the enzyme is present, for example in a solution or composition, at a purity of at least 60, 70, 80, 90, 95 or 99% w/w (dry weight) when assessed relative to the presence of other components, particularly other polypeptide components, e.g. in the solution or composition.

A solution of the enzyme may be analysed by quantitative proteomics to identify the extent of purification of the enzyme of the invention, e.g. to assess if it is the predominant component. For instance, 2D gel electrophoresis and/or mass spectrometry may be used. Such isolated molecules may be present in preparations or compositions as described hereinafter. Alternatively, the extent of purification may more simply be assessed by e.g. SDS-PAGE followed by Coomassie staining to check for contaminants/impurities.

The enzyme of the present invention may be isolated or purified using any technique known in the art. For instance, the enzyme may be produced with an affinity tag such as a polyhistidine tag (His tag), a strep tag, a FLAG tag, an HA tag or suchlike, to enable isolation or purification of the molecule by affinity chromatography using an appropriate binding partner, e.g. a molecule carrying a polyhistidine tag may be purified using Ni²⁺ ions. Alternatively, the enzyme may be isolated or purified by e.g. size-exclusion chromatography or ion-exchange chromatography.

As an alternative to production of the enzyme of the invention in a protein expression system, it may be chemically synthesised in a non-biological system, e.g. liquid-phase synthesis or solid-phase synthesis may be used. An enzyme produced by chemical synthesis (i.e. by a non-biological method), by contrast, is likely to be produced in an isolated form. Thus, no specific purification or isolation step is required for an enzyme of the invention to be considered isolated, if it is synthesised in a manner which produces an isolated molecule.

The enzyme may be provided in a solution or in a composition, e.g. with a suitable solution to maintain viability. The enzyme may also be provided in lyophilised form or immobilised or tethered to other macromolecules or support materials such as alginate beads, iron affinity beads, nickel columns and electrochemical electrodes.

A second aspect of the invention provides a nucleic acid molecule comprising a nucleotide sequence encoding the cytochrome P450 enzyme of the invention. Thus the invention provides a nucleic acid molecule comprising a nucleotide sequence encoding a cytochrome P450 enzyme comprising the amino acid sequence set forth in SEQ ID NO: 3, or a variant thereof having an amino acid sequence having at least 95% identity thereto and having CYP450 activity.

It will be appreciated by those of ordinary skill in the art that, as a result of the degeneracy of the genetic code, there are many nucleotide sequences that may encode any given amino acid sequence. By degenerate nucleotide sequences is meant two (or more) nucleotide sequences which encode the same protein (or protein sequence), specifically in the open reading frame of the reference nucleotide sequence which begins at position 1 (i.e. in which codon 1 of the encoding sequence corresponds to positions 1-3 of the reference nucleotide sequence).

The native nucleotide sequence of the cytochrome P450 enzyme of SEQ ID NO: 3 is set forth in SEQ ID NO: 9. Thus in a particular embodiment the nucleic acid molecule of the invention comprises the nucleotide sequence of SEQ ID NO: 9. In another embodiment, the nucleic acid molecule of the invention comprises a nucleotide sequence that is degenerate with SEQ ID NO: 9, e.g. a codon-optimised version of SEQ ID NO: 9. In another embodiment the nucleic acid molecule of the invention comprises a nucleotide sequence that is a variant of SEQ ID NO: 9, having at least 90, 95, 96, 97, 98 or 99% identity to SEQ ID NO: 9. The nucleic acid molecule may comprise or consist of the stated sequence.

The nucleic acid molecule of the invention may be an isolated nucleic acid molecule and may further include DNA or RNA or chemical derivatives of DNA or RNA. The term “nucleic acid molecule” specifically includes single and double stranded forms of DNA and RNA. Methods for isolating or synthesising nucleic acid molecules are well known in the art.

The invention further provides a construct comprising the nucleic acid molecule of the invention. The construct is conveniently a recombinant construct comprising the nucleic acid molecule of the invention. In the construct, the nucleic acid molecule of the invention may be flanked by restriction sites (i.e. nucleotide sequences recognised by one or more restriction enzymes) to enable easy cloning of the nucleic acid molecule of the invention. In the construct of the invention the nucleotide sequence encoding the enzyme of the invention may conveniently be operably linked within said construct to an expression control sequence, which may be heterologous to the nucleic acid molecule, i.e. non-native, meaning that the expression control sequence and nucleic acid molecule are not found together in any native molecule. Such an expression control sequence is typically a promoter, though the nucleotide sequence encoding the enzyme may alternatively or additionally be operably linked to other expression control sequences such as a terminator sequence, an operator sequence, an enhancer sequence or suchlike. Accordingly, the construct may comprise a native or non-native promoter (relative to the nucleic acid molecule), preferably a non-native promoter. The promoter may be constitutive or inducible.

The term “operatively linked” refers to the association of two or more nucleic acid molecules on a single nucleic acid fragment so that the function of one is affected by the other. For example, a promoter is operatively linked to a coding sequence when it is capable of affecting the expression of that coding sequence (i.e. the coding sequence is under the transcriptional control of the promoter). Coding sequences may be operatively linked to regulatory sequences in sense or antisense orientation.

The term “expression control sequence” refers to nucleotide sequences located upstream (5′ non-coding sequences), within, or downstream (3′ non-coding sequences) of a coding sequence, and which influence transcription, RNA processing or stability, or translation of the associated coding sequence. Expression control sequences may include promoters, operators, enhancers, translation leader sequences, a TATA box, a B recognition element and suchlike. As used herein, the term “promoter” refers to a nucleotide sequence capable of controlling the expression of a coding sequence or RNA. Suitable examples are provided hereinafter. In general, a coding sequence is located 3′ to a promoter sequence. Promoters may be derived in their entirety from a native gene, or be composed of different elements derived from different promoters found in nature, or even comprise synthetic nucleotide segments. It is further recognised that since in most cases the exact boundaries of regulatory sequences have not been completely defined, nucleic acid fragments of different lengths may have identical regulatory activity.

Methods for preparing a construct of the invention are well known in the art, e.g. conventional polymerase chain reaction (PCR) cloning techniques can be used to construct the nucleic acid molecule of the invention which may be inserted into suitable constructs (e.g. containing an expression control sequence) using known methods.

The invention further provides a vector comprising a nucleic acid molecule or construct of the invention. The term “vector” as used herein refers to a vehicle into which the nucleic acid molecule or construct of the invention may be introduced (e.g. be covalently inserted) from which the enzyme or mRNA encoding it may be expressed and/or the nucleic acid molecule/construct of the invention may be cloned. The vector may accordingly be a cloning vector or an expression vector.

The nucleic acid molecule or construct of the invention may be inserted into a vector using any suitable methods known in the art, for example, without limitation, the vector and nucleic acid molecule may be digested using appropriate restriction enzymes and then may be ligated with the nucleic acid molecule having matching sticky ends, or as appropriate the digested nucleic acid molecule may be ligated into the digested vector using blunt-ended cloning.

The vector is generally a prokaryotic, specifically bacterial, vector. The nucleic acid molecule or construct of the invention may be produced in or introduced into a general-purpose cloning vector, particularly a bacterial cloning vector, e.g. an Escherichia coli cloning vector. Examples of such vectors include pUC19, pBR322, pBluescript vectors (Stratagene Inc.) and pCR TOPO® from Invitrogen Inc., e.g. pCR2.1-TOPO.

The nucleic acid molecule or construct of the invention may be sub-cloned into an expression vector for expression of the enzyme of the invention. Expression vectors can contain a variety of expression control sequences. In addition to control sequences that govern transcription and translation, vectors may contain additional nucleic acid sequences that serve other functions, including for example vector replication, selectable markers etc. Plasmids are preferred vectors according to the invention.

The vector of the invention may further comprise a nucleotide sequence encoding a ferredoxin for use with the enzyme of the invention, as required for the enzyme's cytochrome P450 activity. In a particular embodiment, the vector may comprise a nucleotide sequence encoding the ferredoxin of SEQ ID NO: 4 (SeuF08). The native SeuF08 encoding sequence is set forth in SEQ ID NO: 10. In a particular embodiment, the vector comprises the nucleotide sequence of SEQ ID NO: 10, or a nucleotide sequence degenerate with SEQ ID NO: 10.

When the vector of the invention comprises both a nucleic acid molecule of the invention and a nucleotide sequence encoding a ferredoxin, the two genes may be encoded polycistronically, i.e. within an operon such that expression of both genes is controlled by the same promoter. Alternatively, the two genes may be encoded with separate promoters.

Alternatively or additionally, the vector of the invention may further comprise a nucleotide sequence encoding a ferredoxin reductase (e.g. a ferredoxin-NADP⁺-reductase) for use with the enzyme of the invention. Preferably the vector comprises nucleotide sequences (i.e. genes) encoding the enzyme of the invention, a ferredoxin and a ferredoxin reductase. The ferredoxin reductase may be encoded as part of an operon with the enzyme of the invention. In a particular embodiment the enzyme of the invention, ferredoxin and ferredoxin reductase are encoded in a single operon. The genes may be encoded in any order within such an operon.

In a particular embodiment the vector encodes the ferredoxin reductase Scf15A. Scf15A has the amino acid sequence set forth in SEQ ID NO: 11. In a particular embodiment, the vector may comprise a nucleotide sequence encoding the ferredoxin reductase of SEQ ID NO: 11. The native Scf15A coding sequence is set forth in SEQ ID NO: 12. In a particular embodiment, the vector comprises the nucleotide sequence of SEQ ID NO: 12, or a nucleotide sequence degenerate with SEQ ID NO: 12.

The invention further provides a microorganism comprising the nucleic acid molecule of the invention, the recombinant construct of the invention or the vector of the invention, wherein the nucleotide sequence encoding the enzyme of the invention is heterologous to the microorganism; or a lysate of such a microorganism. That is to say, the microorganism of the invention does not natively comprise the nucleotide sequence of the nucleic acid of the invention, and more generally the microorganism of the invention does not natively encode or express the cytochrome P450 enzyme of SEQ ID NO: 3. The microorganism is thus not Streptomyces eurythermus NRRL 2539. Preferably the microorganism is not Streptomyces eurythermus, i.e. it is not any strain of Streptomyces eurythermus. The term “lysate” as used herein is interchangeable with “extract”.

The microorganism is generally a prokaryote, particularly a bacterium. The bacterium may be a Gram-positive or Gram-negative species or strain, generally a non-pathogenic bacterium. In a preferred embodiment, the bacterium is Escherichia coli.

The microorganism may be a cloning host or an expression host. Suitable bacterial expression strains are known, e.g. E. coli expression strains, such as E. coli (DE3) strains.

A lysate (or extract) of the invention (i.e. a lysate or extract of a microorganism of the invention) comprises the enzyme of the invention. Thus the lysate is a lysate of a microorganism that expresses the enzyme (particularly of a bacterium that expresses the enzyme). Such a lysate or extract may be obtained using standard methods of microorganism cell lysis. For instance, the microorganism may be mechanically lysed (e.g. by French press), acoustically lysed (e.g. by sonication), chemically lysed using an appropriate lysis buffer/reagent (e.g. BugBuster, Sigma Aldrich, USA) or lysed by freeze-thaw. The lysate or extract may be a raw lysate/extract, i.e. subjected to no additional treatment following lysis. Alternatively, the lysate may be processed, e.g. the insoluble fraction may be removed (e.g. by centrifugation) such that only the soluble fraction of the lysate is provided. The resulting soluble fraction may be frozen for later use as described below, or in the preferred embodiment the frozen soluble fraction is lyophilised and preferably the container vessels, e.g. vials containing the resulting lyophilisate, are sealed under vacuum. A lysate (or extract) thus generally encompasses a lysate/extract which has been enriched for the enzyme of the invention relative to the raw lysate/extract.

A further aspect of the invention provides the use of the cytochrome P450 enzyme comprising SEQ ID NO: 3, or a variant thereof having at least 95% identity thereto and having CYP450 activity, for the oxidation of an organic compound. In such uses (and other aspects of the invention) the enzyme is preferably a preferred enzyme of the invention as described herein.

Specifically, and in a preferred aspect, the present invention provides the use of the enzyme cytochrome P450_{SeuC10}. This enzyme has the amino acid sequence shown in SEQ ID NO: 3.

The enzyme of the invention and for uses, methods and kits of the invention is present in the strain Streptomyces eurythermus, a deposit of which is held by the Mycotoxin Prevention and Applied Microbiology Research Unit, National Center for Agricultural Utilization Research, Peoria, Illinois, United States of America, under the Accession number NRRL 2539. The strain has also been deposited with various other Culture Collection, with the accession numbers ATCC 14975, ATCC 19749, CBS 488.68, DSM 40014, ETH 6677, IFO 12764, IMET 43078, ISP 5014, JCM 4206, JCM 4575, RIA 1030. When this enzyme, or variants thereof, are combined with suitable reductase components, it is able to oxidise organic compounds.

The enzyme cytochrome P450_{SeuC10} can be extracted, with or without purification from the known Streptomyces eurythermus NRRL 2539, or other bacterial strain, or similarly extracted, with or without purification from a recombinant expression system via cloning of cytochrome P450_{SeuC10} into an expression system, such as E. coli, as will be understood by the skilled person.

Actinomycetes including Streptomyces eurythermus NRRL 2539 readily undergo mutation both through natural causes and as a result of artificial treatments such as UV irradiation, radiation treatment and chemical treatment. The present invention embraces all productive mutants of Streptomyces eurythermus NRRL 2539. These mutant strains also include any strains obtained by gene manipulation such as gene recombination, transduction and transformation. It is also well-known that the properties of actinomycetes change in some degree even for the same strain after successive cultures. Therefore, strains cannot always be differentiated taxonomically because of a slight difference in culture properties. This invention embraces all strains that can produce the cytochrome P450 enzyme, and especially strains that cannot be clearly differentiated from strain NRRL 2539 or its mutants.

One skilled in the art will appreciate that the present invention can include variants of the particular amino acid sequence which is exemplified herein. Particularly preferred are variants having an amino acid sequence similar to that of the amino acid sequence disclosed herein, in which one or more amino acid residues are substituted, deleted or added in any combination. Especially preferred are silent substitutions, additions and deletions, which do not alter the properties and activities of the protein of the present invention. Various amino acids have similar properties, and one or more such amino acids of a substance can often be substituted by one or more other amino acids without eliminating a desired activity of that substance. Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). The above described substitutions are considered conservative substitutions. Variants include naturally occurring and artificial variants. Artificial variants may be generated using mutagenesis techniques, including those applied to nucleic acid molecules, cells or organisms. Preferably, the variants have substantial identity to the amino acid sequence exemplified herein, as mentioned hereinbefore. As used herein, the term “variant” or “mutant thereof” refers to amino acid sequences which have “substantial identity”, preferably having at least 95%, 96%, 97%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.1%, 99.8% or 99.9% identity with SEQ ID NO: 3. Desirably, the term “substantial identity” indicates that said sequence has a greater degree of identity with any of the sequences described herein than with prior art amino acid sequences. One can use a program such as the online tool using the CLUSTAL W algorithm (Thompson, J. D., Higgins, D. G. and Gibson, T. J. (1994). Nucleic Acids Research, 22:4673-4680.) to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. The above applies mutatis mutandis to all amino acid sequences disclosed in the present application. Nucleic acid sequences may be similarly aligned, and their sequence identities calculated, using any suitable programme, e.g. Emboss Needle, e.g. in relation to aspects of the invention concerning nucleic acid sequences.

In a preferred embodiment, the term “variant” generally refers to a sequence having at least 95% identity to SEQ ID NO: 3 and also having CYP450 activity, more preferably at least 96% identity thereto or at least 97% identity thereto, further preferably 98% identity thereto, even more preferably 99% identity thereto, most preferably 100% identity thereto. A variety of different compounds can be oxidised (e.g. hydroxylated, dealkylated, epoxidated, etc.) using the claimed cytochrome P450 enzyme. In a preferred embodiment, the organic compound to be oxidised will have a rate of conversion to the resulting derivative of at least 3%, more preferably at least 5%, more preferably at least 10%, more preferably at least 25%, more preferably at least 50%, even more preferably at least 70% and most preferably a rate of conversion to the resulting derivative of 100%, using the same conditions described in Example 4 herein.

The compound to be oxidised by the cytochrome P450 enzyme may have an optionally substituted or unsubstituted linear or branched alkyl group, such as methyl, isopropyl or tert-butyl, which is hydroxylated; or an aromatic group, such as an optionally substituted aryl or heteroaryl, which is hydroxylated; or an olefinic group, or substituted aryl or heteroaryl, which is epoxidated; or an alkyl-heteroatom, which is dealkylated.

There is a particularly high conversion rate for these reactions when using the claimed cytochrome P450 enzyme.

Preferably, the compound to be oxidised is of formula I:

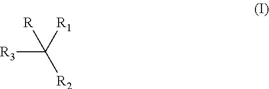

where R represents the rest of the compound, and where R¹, R² and R³ are independently selected from H or C₁₋₁₂ alkyl or C₆₋₁₀ aryl, or wherein any two of R¹, R² and R³ may be joined to form an optionally substituted cycloalkyl or heterocycloalkyl or R¹, R² and R³ may be joined together with their bridging carbon to form an olefin, aryl or heteroaryl.

Preferably R is an optionally substituted alkyl; an optionally substituted olefin, an optionally substituted aryl, optionally substituted heteroaryl or optionally substituted heterocycloalkyl.

As used herein “alkyl” means a C₁-C₁₀ alkyl group, which can be linear or branched or cyclic. Examples include propyl and butyl, pentyl, hexyl, cyclopentyl and cyclohexyl. Preferably, it is a C₃-C₁₀ alkyl moiety. More preferably it is a C₅-C₆ alkyl moiety. Preferably the alkyl is an optionally substituted cyclohexyl.

For the avoidance of any doubt, the term cycloalkyl is a cyclic alkyl group.

As used herein “aryl” means an optionally substituted monocyclic, bicyclic or tricyclic aromatic radical, such as phenyl, biphenyl, naphthyl, anthracenyl. Preferably the aryl is an optionally substituted C₆ aryl.

As used herein “heteroaryl” means an optionally substituted monocyclic, bicyclic or tricyclic aromatic radical containing at least one and up to four heteroatoms selected from oxygen, nitrogen and sulfur, such as furanyl, pyrrolyl, thiazolyl, isothiazolyl, tetrazolyl, imidazolyl, oxazolyl, isoxazolyl, thienyl, pyrazolyl, pyridinyl, pyrazinyl, pyrimidinyl, indolyl, azaindolyl, isoindolyl, quinolyl, isoquinolyl, triazolyl, thiadiazolyl, oxadiazolyl.

As used herein heterocycloalkyl means an optionally substituted cycloalkyl wherein one to four carbon atoms have been substituted with a heteroatom. Preferably, the heteroatoms are selected from nitrogen, oxygen, sulphur or phosphorous.

As used herein the term “optionally substituted” means an H has been removed from a compound and replaced with an organic fragment such as those comprising a combination of any of carbon, halogen, hydrogen, nitrogen, oxygen and sulphur.

Preferably the compound of formula I has a molecular weight of from 50 to 1000, such as from 100 to 700, more preferably from 200 to 500.

Preferably, R¹, R² and R³ are independently selected from H, C₁₋₆ alkyl or C₆₋₁₀ aryl, preferably with the proviso that either one or more of R¹, R² and R³ is H. Most preferably, R¹, R² and R³ are independently selected from H, methyl, ethyl, propyl, butyl, t-butyl, pentyl and hexyl preferably with the proviso that either one or more of R¹, R² and R³ is H.

In a particularly preferred embodiment, the cytochrome P450 enzyme is reacted with a compound such as carbamazepine, bosentan, diclofenac, meloxicam, tivantinib or ambroxide. Other preferred compounds to be used as the substrate are as set out in the Examples, particularly ambroxide, diclofenac, tivantinib, carbamazepine, palmitic acid, BIRB796, vanoxerine, ruxolitinib and perindopril.

The preferred compounds to be oxidised are typically of the following structural formulae:

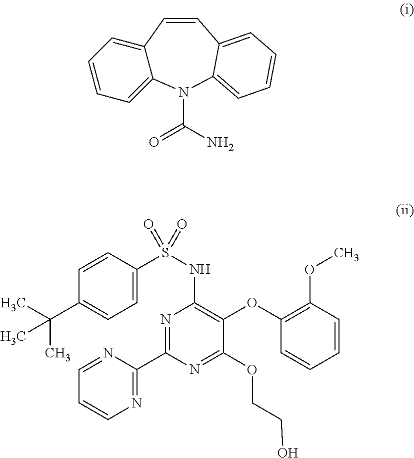 [SMILES: NC(=O)N1c2ccccc2C=Cc2ccccc21 ; COc1ccccc1Oc1c(NS(=O)(=O)c2ccc(C(C)(C)C)cc2)nc(-c2ncccn2)nc1OCCO]

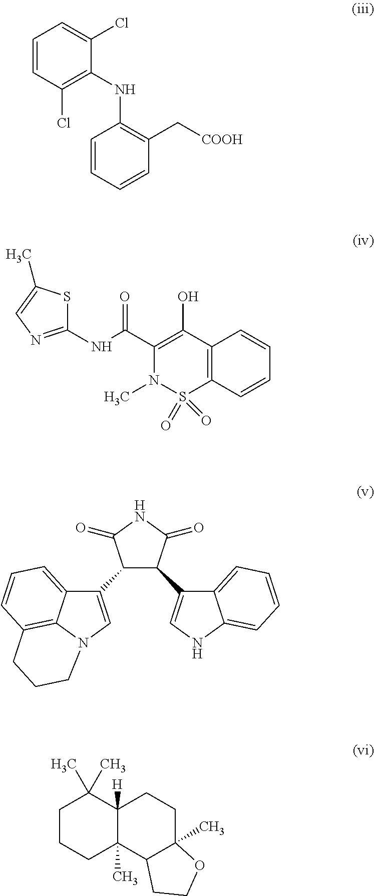 [SMILES: O=C(O)Cc1ccccc1Nc1c(Cl)cccc1Cl ; Cc1cnc(NC(=O)C2=C(O)c3ccccc3S(=O)(=O)N2C)s1 ; O=C1NC(=O)[C@@H](c2cn3c4c(cccc24)CCC3)[C@@H]1c1c[nH]c2ccccc12 ; CC1(C)CCC[C@]2(C)C3CCO[C@]3(C)CC[C@@H]12]

The cytochrome P450 enzyme may optionally be used in combination with reductase components, which activate the cytochrome P450. In a preferred embodiment, ferredoxin and ferredoxin reductase components are used. Any components which activate the cytochrome P450 may also be used, including those fused directly or by peptide linkage, or chemical-based oxygen providing surrogates such as peroxide, iodane or chemicals of similar resulting properties. In a particularly preferred embodiment, the enzyme cytochrome P450_{SeuC10} having SEQ ID NO: 3 or a variant thereof having at least 95% identity thereto and having CYP450 activity, is combined with suitable ferredoxin and ferredoxin reductase components to give an effective system to convert a substrate compound to a resulting oxidised derivative.

In a preferred embodiment, the cytochrome P450 enzyme or variant thereof is present in Streptomyces eurythermus NRRL 2539 cells.

In another preferred embodiment, the cytochrome P450 enzyme or variant thereof is expressed by at least one recombinant microorganism comprising heterologous nucleic acid encoding the enzyme, derived from Streptomyces eurythermus NRRL 2539. As used herein the term “comprising” is intended to mean containing at least the claimed sequence, but may include other sequences. In one embodiment, the recombinant microorganism comprises a heterologous nucleic acid encoding the enzyme or variant thereof. In an alternative embodiment, the recombinant microorganism also comprises a heterologous nucleic acid encoding a reductase agent. “Heterologous” has the meaning as described hereinbefore, i.e. the microorganism does not natively comprise the nucleotide sequence of the nucleic acid of the invention.

In another aspect of the invention, there is provided a method for the production of an oxidised organic compound, comprising reacting the organic compound with a cytochrome P450 enzyme comprising SEQ ID NO: 3 or a variant thereof having at least 95% identity thereto and having CYP450 activity.

The choice of compound to be oxidised is discussed above.

In a preferred embodiment, the enzyme is used to catalyse the oxidation of an alkyl or aryl group.

In a particularly preferred embodiment, the compound to be oxidised is carbamazepine, bosentan, diclofenac, meloxicam, tivantinib or ambroxide, or derivatives thereof or other compounds as described in the Examples and hereinbefore.

Optionally, one or more additional component(s) may be used to activate the cytochrome P450 enzyme. In an embodiment according to the present invention, the cytochrome P450 enzyme of the invention is used in combination with reductase components, preferably with ferredoxin and ferredoxin reductase components.

In an embodiment of the invention, the enzyme is present in a host cell, i.e. a host cell is used for biotransformation of the substrate compound. In a preferred embodiment of the invention, the cytochrome P450 enzyme or variants thereof are present in Streptomyces eurythermus NRRL 2539 cells (i.e. the host cell is a Streptomyces eurythermus NRRL 2539 cell). In another preferred embodiment the host cell is a microorganism of the invention (as described above). The cells may be dosed with the organic compound to be oxidised. The method may optionally comprise an additional step wherein the cells are subsequently harvested and purified (or isolated) to obtain the oxidised compound. In particular, the cells are subsequently harvested and the oxidised compound isolated. That is to say, the method may optionally comprise an additional step in which the oxidised compound is purified (or isolated). In this step the cells are first harvested (e.g. by centrifugation). If the oxidised compound is secreted by the cells, such that it is present in the cell supernatant, the oxidised compound is extracted from the supernatant. Such extraction may be performed using standard methods in the art, as discussed below.

If the oxidised compound is present in the cells, the cells may be lysed (or extracted), e.g. using methods as described above, and the oxidised compound isolated from the lysate. Such isolation may be performed by standard methods in the art, as discussed below.

Culture of the Streptomyces eurythermus NRRL 2539 to produce the P450 enzyme extracts is suitably performed by seeding of a conventional culture medium containing nutrients well-known for use with such microorganisms. Thus, the culture medium contains sources of assimilable carbon and of assimilable nitrogen. The culture medium may also contain inorganic salts. Examples of sources of assimilable carbon include glucose, sucrose, starch, glycerin, millet jelly, molasses and soybean oil. Examples of sources of assimilable nitrogen include soybean solids (such as soybean meal or soybean flour), wheat germ, meat extracts, peptone, corn steep liquor, dried yeast and ammonium salts, such as ammonium sulphate. If required, inorganic salts, such as sodium chloride, potassium chloride, calcium carbonate and various phosphates, may also be included. The medium is preferably sterilized and has a pH adjusted to 5 to 8. Culture of any other bacterial strain or species may similarly be performed in any appropriate medium, as known in the art.

The skilled person will understand that the particular cultivation technique employed is not critical to the invention and any technique commonly used for the cultivation of Actinomycete bacteria (or other types of bacteria as required) may equally be employed with the present invention. In general, the techniques employed will be chosen having regard to industrial efficiency. Thus, liquid culture is generally preferred and the submerged culture method is most convenient from the industrial point of view. Cultivation is preferably carried out under aerobic conditions.

The enzyme of this invention may be produced with an induction agent present. For preference, but not limited to, the induction agent is selected to be the same as the intended substrate for the isolated enzyme. When from 4 hours to 3 days have elapsed after inoculation, preferably 0.05 to 5 mM, more preferably 0.2 mM of induction agent is added, and then cultivation is continued for 2 hours to 1 week, preferably for about one day. The temperature of cultivation is typically 20° C. to 45° C., preferably 25° C. to 30° C., optimally about 27° C. Shake culture or aeration techniques can be adopted.

The cells obtained by the cultivation may be disrupted by cell disruption techniques such as high-pressure homogenisation in buffer solution. The supernatant obtained by centrifugation gives the crude enzyme solution. For example, the enzyme of the present invention can be obtained in a supernatant produced by centrifugation at 38,000×g for 20 minutes.

In an alternative embodiment, the cytochrome P450 enzyme or variants thereof are expressed by at least one recombinant microorganism comprising a heterologous nucleic acid encoding the enzyme (i.e. a heterologous nucleic acid derived from Streptomyces eurythermus NRRL 2539).

Here, the at least one recombinant microorganism can be dosed with an organic compound to be oxidised. This method may optionally comprise an isolation and/or purification step(s) to obtain the oxidised compound, as described above.

In a preferred embodiment, this can be achieved by the recombinant expression of the functional cytochrome P450_{SeuC10} with intact haem. This can be expressed with any or all of the cofactor enzymes. In a particularly preferred embodiment, ferredoxin and ferredoxin reductase may be expressed. This can be achieved by polycistronic plasmid use or via fusion, either via linkers or directly into a single protein product.

Alternatively, the functional cytochrome P450_{SeuC10} protein may be expressed alone without mixing with cofactor enzymes. In a preferred embodiment, cofactor enzymes may be titrated in to provide the active enzyme reaction after material production. The cofactors may be obtained by extraction from wild-type or recombinant materials derived from plants or microbial fermentation. Hussain & Ward, Appl Environ Microbiol. 2003; 69(1):373-382, describe exemplary cloning techniques that may be used.

The native organism, host strain expressing the recombinant enzyme or extracted enzyme is contacted directly with the substrate, preferably in an aqueous medium, either mono or biphasic. Reaction conditions, including choice of pH and temperature will be evident to the skilled person, based on conventional techniques. For example, the reaction may be performed at a pH value in the range of from 5 to 11, more preferably 6.5 to 9.0, most preferably around 8 may be used. To achieve this pH, a selected microbial growth medium or phosphate buffer solution may be used which has the above-mentioned pH. The reaction temperature is preferably within the range from 20° C. to 45° C., more preferably from 25° C. to 30° C. The concentration of the substrate in the reaction medium is preferably within the range from 0.01 to 5.0% by weight. The time allowed for the reaction is normally from 1 minute to 5 days, more usually from 1 day to 5 days, although this may vary, depending upon the concentration of substrate in the reaction mixture, the reaction temperature, and other factors. The extracted enzyme material can either be used directly after extraction, or after storage in frozen solution. In a particularly preferred embodiment, the extracted enzyme material can be dried, preferably by lyophilisation, with or without vessel closure under vacuum, for later use with or without the addition of other components required for reaction, such as other enzyme cofactor components.

After completion of the conversion reaction, the resulting oxidised compound can be isolated (or purified) using conventional procedures, including, for instance, filtration, solvent extraction, chromatography, crystallization, and other isolation procedures. Such procedures will be selected having due regard to the identity of the product. Before, during or after the isolation, the product may or may not be derivatised, as desired. Isolation and purification are referred to herein, in some cases interchangeably. Isolation may be considered a form of purification or the first step of purification, e.g. separation of the cell or oxidised compound from the bulk reaction. Further purification steps may then be conducted to achieve improved purity. Thus reference to isolation herein may be considered a first purification step. Purification may comprise only a first step of isolation, but may also include additional steps to achieve higher levels of purity. Preferably purity levels of at least 80, 85, 90 or 95% w/w (dry weight) are achieved for the oxidised compound.

The starting materials as substrates for the enzyme may be either derived from synthetic routes, naturally occurring, either via natural biomass such as plant material, or produced by fermentation, or by mixed routes thereof. Enzyme reactions can also be performed using pure or non-purified materials, the resulting reaction may be used to aid later purifications of reacted or unreacted components.

Of the substrate compounds used as starting materials, free bases, alkali metal salts, e.g. the sodium or potassium salts, or acid salts of organic or inorganic nature such as tosylate or hydrochlorides, are suitable for use.

After completion of the conversion reaction, the desired compound can be obtained from the reaction system, collected, isolated and purified by conventional means if required, or onward used directly in unpurified form. For example, the reaction product may be centrifuged or filtered and the supernatant or filtrate extracted with a hydrophobic resin, ion-exchange resin or water-immiscible organic solvent such as ethyl acetate. After evaporation of the solvent of the extract, the remaining crude material, for example the remaining crude oxidised compound, may be purified by subjecting it to column chromatography using silica gel or alumina or reversed-phase stationary phase, and by eluting with a suitable eluent. If the starting material is a mixture, then the product can be isolated as a mixture of oxidised compounds which if desired can be separated using chromatography or other suitable techniques.

In general, the resulting oxidised compound may have improved pharmaceutical or agrochemical properties, such as bioactivity potency, improved solubility characteristics, reduced off-target interactions, or simply be of further utility, such as for onward synthesis, or be useful for an analytical standard.

When the cytochrome P450 enzyme preparations of this invention are reacted with substrate compound at pH 8.0 for 5 minutes with (a) ferredoxin, (b) ferredoxin-NADP⁺-reductase, (c) NADPH regeneration system, and (d) dissolved oxygen, the temperature of reaction ranges at least from 4° C. to 60° C. The optimum pH for each cytochrome ranges from 6.5 to 8.0. Each cytochrome is stable when kept for 24 hours at 4° C. in the pH range between 6.0 and 9.0. Stored lyophilised enzyme is stable at 20-27° C. for 10 days compared to a control stored at <−18° C.

The use of ferredoxin, ferredoxin-NADP⁺-reductase, oxygen and NADPH is not essential. Any components which can activate the cytochrome P450 may be adopted.

Measurement of the enzyme activity is normally effected in one of two ways:

### (i) Measurement on Cytochrome P450:

Measurement is performed according to the method of Omura and Sato et al. (J Biol Chem, 239. 1964, 2370). That is to say, cytochrome P450 is analyzed quantitatively using the following formula, based on the difference in the absorbance of the reduced CO versus the reduced difference spectrum at 450 nm and 490 nm.

$$Cytochrome P 450 ( mM ) = \frac{Abs ( 450 nm ) - Abs ( 490 nm )}{91 ( mM {cm}^{- 1} ) \times l ( cm )}$$

(ii) Measurement of Rate of Formation of Oxidised Substrate Compound from Substrate Compound

The following cocktail of components is employed:

| | |
|---|---|
| Potassium phosphate buffer pH 8.0 | 100 mM |
| MgCl₂ | 5 mM |

Enzyme solution containing expressed FdX, FdR, P450 Native concentration when pellet extracted at a rate of 0.30 g cell wet weight per ml extraction buffer

| | | |
|---|---|---|
| NADP⁺ | 1 | mM |
| Glucose-6-phosphate | 5 | mM |
| Glucose-6-phosphate dehydrogenase | 1 | UN/ml |
| Substrate compound | 0.1 | mg/ml |

| | |
|---|---|
| Total volume | e.g., 0.1-0.5 ml |

To measure enzyme activity the components of the table are mixed, the solution is shaken at 27° C. for 16-20 hours, and then e.g., 100-500 μl of ACN is added and the reaction stopped. The amount of oxidised substrate formed by the enzyme system is determined with HPLC or UPLC. The reaction may be used on a preparative scale by increasing the volume as appropriate.

In a further aspect, the invention provides a kit comprising:
i) a cytochrome P450 enzyme comprising SEQ ID NO: 3 or a variant enzyme having at least 95% identity thereto and having CYP450 activity;
ii) a microorganism that expresses a cytochrome P450 enzyme comprising SEQ ID NO: 3 or a variant enzyme having at least 95% identity thereto and having CYP450 activity, or a lysate of said microorganism; and/or
iii) a nucleic acid molecule, recombinant construct or vector of the invention as defined above.

Most preferably the kit comprises the cytochrome P450 enzyme of the invention.

If a kit comprises a microorganism or lysate (either fresh, frozen and/or lyophilised) thereof, preferably the microorganism is a microorganism of the invention, as described above, or a lysate of a microorganism of the invention. The kit components, such as the cytochrome P450 enzyme, microorganism or lysate, or nucleic acid molecule, recombinant construct or vector may be lyophilised and/or vacuum sealed.

The kit may further comprise instructions for use for the oxidation of an organic compound. The kit allows the user to screen for the oxidation of compounds of interest.

In a preferred embodiment, the kit further comprises electron donating agents. This is particularly advantageous when the kit comprises the enzyme of the invention. The kit preferably comprises as the electron donating agents a ferredoxin reductase and a ferredoxin with cofactors NADH or NADPH or cofactor regeneration systems such as NAD+ or NADP+, glucose or glucose-6-phosphate, and glucose-dehydrogenase or glucose-6-phosphate dehydrogenase. However, any suitable electron donating agents may be used.

Optionally, the kit may further comprise a buffer, either separately or contained with the other components. This is particularly advantageous when the kit comprises the enzyme of the invention.

Preferably, the kit may further comprise one or more other CYP450 enzymes. This is particularly advantageous when the kit comprises the enzyme of the invention. When the kit comprises a microorganism expressing the enzyme of the invention, the microorganism may further express one or more additional CYP450 enzymes, or the kit may comprise one or more additional microorganisms, or their lysates, each of which expresses a further CYP450 enzyme (i.e. a CYP450 enzyme other than that of the invention). When the kit comprises a nucleic acid molecule, recombinant construct or vector encoding the enzyme of the invention, the nucleic acid molecule, recombinant construct or vector may further encode one or more additional CYP450 enzymes, or the kit may further comprise one or more additional nucleic acid molecules, recombinant constructs or vectors, each of which encodes a further CYP450 enzyme (i.e. a CYP450 enzyme other than that of the invention).

Preferably, the cytochrome P450 enzyme or microorganism or its lysate is lyophilised or immobilised or tethered to other macromolecules or support materials such as alginate beads, iron affinity beads, nickel columns and electrochemical electrodes.

In a further aspect the invention provides a method of producing a cytochrome P450 enzyme of the invention, the method comprising introducing a nucleic acid molecule, recombinant construct or vector of the invention into a microorganism, and expressing the cytochrome P450 enzyme in the microorganism, and optionally purifying (or isolating) the cytochrome P450 enzyme.

Techniques for performing the method are well known in the art. The nucleic acid molecule, recombinant construct or vector may be generated using standard techniques, as described above. The microorganism into which the nucleic acid molecule, construct or vector is introduced is preferably as described above in the context of the microorganism of the invention. That is to say, the microorganism is preferably a bacterium, e.g. E. coli. The enzyme is expressed in the microorganism using standard techniques in the art (e.g. as demonstrated in the Examples below).

To obtain active enzyme the microorganism may be lysed, to provide a lysate comprising the enzyme. Lysis may be performed using standard methods in the art, e.g. French press. The enzyme may then be purified (or isolated), if desired. Purification may be performed using standard methods in the art. For example, the enzyme may be expressed with an affinity tag (e.g. a His tag or a Strep tag) and then purified by affinity chromatography, as described above.

The methods of the present invention are demonstrated in the examples below. These examples are provided as an illustration only and should not be construed as limiting on the present invention.

### EXAMPLES

### Example 1: Cloning of P450_{SeuC10} from Streptomyces eurythermus NRRL 2539

Extraction of Genomic DNA from Streptomyces eurythermus NRRL 2539

Genomic DNA (gDNA) was isolated from cell pellet of fermentation material of Streptomyces eurythermus NRRL 2539. Culture medium containing 4 g/L yeast extract; 10 g/L malt extract; 4 g/L glucose and adjusted to pH 7.0. Two Erlenmeyer flasks of 250 ml volume, each of which contained 50 ml of the medium, were sterilized 115° C. for 20 minutes. Streptomyces eurythermus NRRL 2539 was recovered from cryovial stocks stored in liquid nitrogen and inoculated into the two flasks containing 50 ml of the above growth medium. After 2 days of growth at 27° C. and 200 rpm, 50 mls of culture were transferred to 50 ml centrifuge tubes and centrifuged to collect the pelleted cells. The pellet was washed once with an isotonic buffer to remove residual medium components before freezing the pellet at −80° C. for later extraction of genomic DNA as described below. The cell pellet was defrosted and resuspended in 7.5 ml TE buffer (10 mM Tris-HCl PH 7.5, 1 mM Na₂EDTA). Seventy-five μl of 20 mg/ml lysozyme solution was added and the solution was incubated at 37° C. for 1 hour, followed by addition of 750 μl of 10% (w/v) SDS and mixing by inverting. After addition of 20 μl of 20 mg/ml pronase and incubation at 37° C. for 1.5 hours, the solution was supplemented with 16 μl of 10 mg/ml RNase solution, followed by another incubation step at 37° C. for 1 hour and 50° C. for 1 hour. Nine hundred μl of 0.5 M NaCl solution was added before the solution was extracted twice with an equal volume of phenol:chloroform:isoamyl-alcohol (25:24:1; Sigma-Aldrich). The aqueous layers were collected and gDNA was precipitated with 1 volume of isopropanol and centrifugation (10,000×g, 30 min, 20° C.). The gDNA pellet was washed once with 100% ethanol and twice with 70% ethanol (˜30 ml each wash step). The gDNA pellet was air-dried and resuspended in 5 ml TE buffer. Concentration and purity of the gDNA was measured using a NanoDrop instrument (Thermo Scientific) and gDNA integrity was assessed by agarose gel electrophoresis.

### PCR Reactions

The P450_{SeuC10} and ferredoxin_{seuF08} gene operon (SEQ ID NO: 1) was cloned from Streptomyces eurythermus NRRL 2539 in a total reaction volume of 50 μl using primers SeuC10-SeuF08_f (5′-primer sequence-3′: ATTTTGTTTAACTTTAAGAAGGAGATATACATATGAAGATCGGCACGACGCACCTC) (SEQ ID NO: 5) and SeuC10-SeuF08_r (5′-primer sequence-3′: CTACCCGCAGAGGGGGGGGCATAAGCTTCCTATTAGGCGGAGCGCTCCCGTACGGTG ATG) (SEQ ID NO: 6). PCR reactions contained 10 μl of 5×GC Green buffer (Thermo Scientific), 2.5 μl of DMSO (Sigma), 10 μL of 5 M betaine (Sigma) and 1 μL of formamide (Sigma), 1 μl of 10 mM of dNTPs (Thermo Scientific), 1 unit of HotStart II Phusion® High-Fidelity DNA Polymerase (Thermo Scientific), ˜90 ng of genomic DNA, 0.5 UM of each forward and reverse primer and the reaction was filled up to a total volume of 50 μl with MilliQ®-H₂O. PCR reactions were performed on an Eppendorf Mastercycler ep Gradient system with the following cycling conditions: 98° C. for 2 minutes, 35 cycles (98° C. for 45 seconds, 72° C. for 30 seconds, 72° C. for 3 minute), 72° C. for 15 minutes. The PCR reaction was analysed by agarose gel electrophoresis and products were extracted from the agarose gel using the Qiagen QIAquick 96 PCR Purification Kit. The concentration of the expected 1558 bp amplicon was measured using the Biochrome Genequant 1300 instrument and on the Molecular Devices Spectramax 384 plus plate reader.

Construction of pHD05 Vector

The pHD05 vector is a derivative of pHD02 (See WO 2018/091885) containing the cer sequence. The cer sequence was amplified from pKS450 plasmid (Summers and Sherratt, EMBO J. 1988; 7 (3): 851-858.) by PCR using the primers ser_f (5′-primer sequence-3′: GGGTCCTCAACGACAGGAGCACGATCATGCCGGAAATACAGGAACGCACGCTG) (SEQ ID NO: 7) and ser_r (5′-primer sequence-3′: TTATCGCCGGCATGGCGGCCCCACGGGTGCCGGGGCACAACTCAATTTGCGGGTAC) (SEQ ID NO: 8). The expected 439 bp amplicon was extracted from the agarose gel using the Thermofisher GeneJet Gel Extraction Kit and cloned into the FspAI site of pHD02 by Gibson assembly. The plasmids containing the cer sequence were analysed by PCR screening and DNA sequence was confirmed by Sanger sequencing at LGC Genomics (Germany). The plasmid containing the cer sequence was designated as pHD05.

Cloning of the P450_{SeuC10} and ferredoxin_{SeuF08} Gene Operon into pHD05 Plasmid

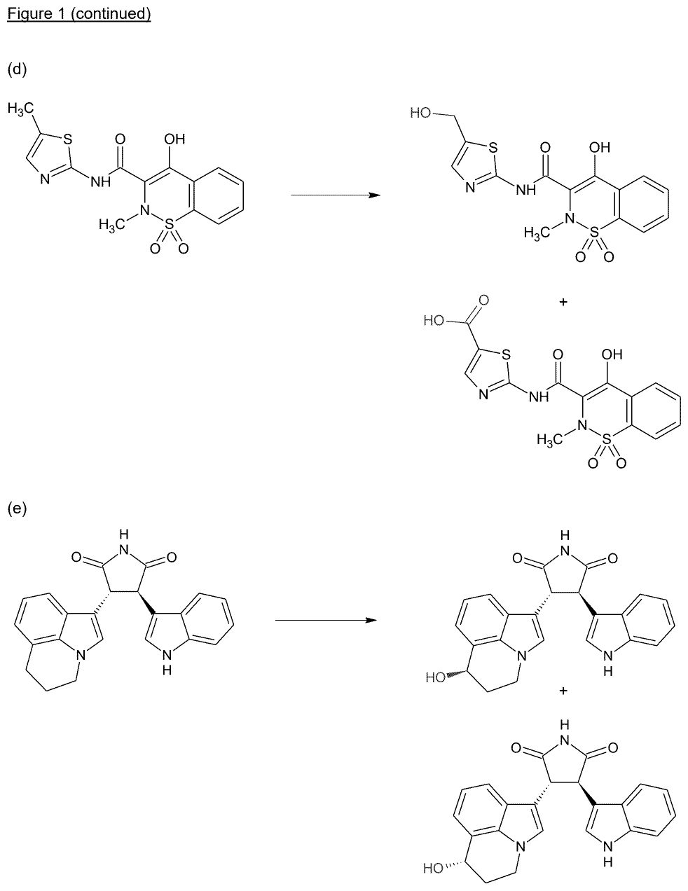

The purified P450_{SeuC10} and ferredoxin_{seuF08} amplicon was assembled into pHD05 vector digested with NdeI and EcorI, so that that the cytochrome P450 and ferredoxin gene operon is introduced into a polycistronic operon containing a ferredoxin reductase (scf15a). The vector was digested with restriction endonuclease (New England Biolabs). Restriction digestion was carried out for 16 h at 37° C. in a total volume of 200 μl containing 20 μl of 10× CutSmart Buffer®, 4 μl of each restriction endonuclease (40 units; New England Biolabs), ˜10.4 μg of plasmid DNA. The reaction was stopped by inactivation of the restriction endonuclease at 65° C. for 20 min. The expected digested products were purified using the Thermo Scientific GeneJET Gel Extraction Kit. The purified digested vector and purified P450 amplicon were assembled together using Gibson assembly in a total volume of 20 μL containing ˜50 ng of digested vector and 1:3 (vector:insert) molar concentration of insert, 6.65% PEG 8000, 133 mM Tris-HCl (Fisher), pH7.5, 13.3 mM MgCl₂ (Sigma), 13.3 mM DTT (Sigma), 0.266 mM dNTP (New England Biolabs), 1.33 mM NAD (New England Biolabs), 0.495 Unit of Phusion DNA polymerase (New England Biolabs), 79.5 Units of Taq DNA ligase (New England Biolabs) and 0.075 Units of T5 exonuclease (New England Biolabs). The reaction mixture was incubated at 50° C. for 1 hour and 1 μL was introduced into 25 μL of chemically competent cells E. coli DH5α (Invitrogen) by chemical transformation. Clones were selected on Miller's Luria broth (LB) plates containing 50 μg/mL kanamycin after 16 hours of incubation at 37° C. Clones were picked and cultivated in 5 mL LB containing the same antibiotic and recombinant plasmids were isolated from the cultures using the QIAGEN QIAprep 96 Plus Kit. DNA sequences of the P450, ferredoxin and ferredoxin reductase were analysed by PCR screening and DNA sequence was confirmed by Sanger sequencing at LGC genomics (Germany). The constructed plasmid was designated as pHD05-SeuC10-SeuF08 (FIG. 2).

### Construction of the Recombinant Expression Strain

The strain E. coli Tuner (DE3) (Merck) was used as a host for recombinant expression of P450_{SeuC10}, ferredoxin_{seuF08} and ferredoxin reductase_{SCF15A}. To construct this expression strain, E. coli Tuner (DE3) cells were transformed with the expression plasmid using chemical transformation. Twenty-five μl of chemically competent cells were mixed with 1 μl (˜100 ng) of pHD05-SeuC10-SeuF08 plasmid followed by incubation on ice for 30 min. Heat shock was performed at 30 sec in a water bath at 42° C. and cells were subsequently chilled on ice for 2 min. One millilitre of LB was added to the cells and incubated for 1 hour at 37° C. and shaking at 250 rpm. The transformation mixture was plated onto LB plates containing 50 μg/ml kanamycin. Plates were incubated at 37° C. for 16 hours. To prepare glycerol stocks of this expression strain, several colonies were picked with a sterile loop and inoculated into 5 ml LB media containing the same antibiotics and cultivated at 37° C. and 250 rpm for 16 h. Five hundred millilitres of this culture were mixed with 500 μl of 50% (w/v) glycerol in cryovials and stored at −80° C.

### Example 2: Expression of Recombinant P450_{SeuC10}

Preculture: Five milliliters of LB Miller media (Sigma) supplemented with 50 μg/ml of kanamycin was inoculated with a loop scraped from a cryovial containing E. coli Tuner (DE3) harbouring the pHD05-SeuC10-SeuF08 expression plasmid. Cells were grown overnight at 37° C. and 250 rpm in a New Brunswick Scientific Innova 4230 shaking incubator. Seed: Into a 250 ml baffled flask, 50 ml of PCM8.1 media supplemented with 50 μg/ml of kanamycin was inoculated with the overnight preculture to an OD600 of 0.1 and incubated at 37° C. and 200 rpm until the end of the day.

The components of PCM8.1 were MgSO₄ (0.49 gL⁻¹), Na₂HPO₄*7H₂O (6.7 gL⁻¹), KH₂PO₄ (3.4 gL⁻¹), NH₄Cl (2.68 gL⁻¹), Na₂SO₄ (0.71 gL⁻¹), arginine (0.2 gL⁻¹), histidine (0.15 gL⁻¹), lysine (0.2 gL⁻¹), phenylalanine (0.2 gL⁻¹), serine (0.2 gL⁻¹), threonine (0.2 gL⁻¹), tryptophan (0.2 gL⁻¹), methionine (0.2 gL⁻¹), monosodium glutamate (8 gL⁻¹), glucose (0.5 gL⁻¹), glycerol (10 gL⁻¹) and a 1000-fold diluted trace element solution with FeCl₃ (81.1 gL⁻¹), CaCl₂*6H₂O (4.38 gL⁻¹), MnCl₂*4H₂O (1.98 gL⁻¹), ZnSO₄*7H₂O (2.88 gL⁻¹), CoCl₂*6H₂O (0.48 gL⁻¹), CuCl₂*2H₂O (0.34 gL⁻¹), NiCl₂*6H₂O (0.48 gL⁻¹), Na₂MoO₄*2H₂O (0.48 gL⁻¹), Na₂SeO₃ (0.35 gL⁻¹), and H₃BO₃ (0.12 gL⁻¹).

Production: A 1 L baffled flask containing 200 mL of PCM8.1 media supplemented with 50 μg/ml of kanamycin, 23.8 μg/ml of IPTG, 320 μg/ml of 5′-aminolevulinic acid and 55 μg/ml of FeSO₄*7H₂O was inoculated with the seed cultures to an OD of 0.6. The induced production cultures were incubated at 27° C. and 200 rpm until the cultures had reached stationary phase (approximately 16-20 hours). The cultures were harvested by centrifugation at 3,000 rpm for 15 minutes. The pellets were washed with 30 mL of wash buffer (isotonic 0.85% NaCl with 5% glycerol) and transferred into a fresh 50 mL centrifuge tube. The cells were further centrifuged at 4,000 rpm for 25-35 minutes and the pellet was stored at −20° C. for processing.

### Example 3: Extraction & Processing of Enzyme Materials

Suspended cell pellets were provided as described in Example 2, containing recombinant P450, ferredoxin and ferredoxin reductase in 100 mM potassium phosphate buffer pH 8.0, 5 mM MgCl₂, 5 mM TCEP, and 1 mM PMSF in a ratio of 3.0 ml of buffer per 1 g of cells. Lysed cells were produced by high pressure disruption using three cycles of 30 kpsi. Lysed material was centrifuged at 38,000×g for 40 minutes (4° C.) and the supernatant was sterilized by passing through a 0.2 micron filter to provide the cell-free enzyme preparation containing recombinant P450, ferredoxin and ferredoxin reductase. The crude extract was then either used immediately for the desired reaction or dispensed into glass vials (typically 0.5 ml per 2 ml vial or 10 ml per 20 ml vial), frozen and lyophilised using an Edwards Supermodulyo Freeze-dryer before being stored in a standard laboratory freezer at −20° C. until required for use.

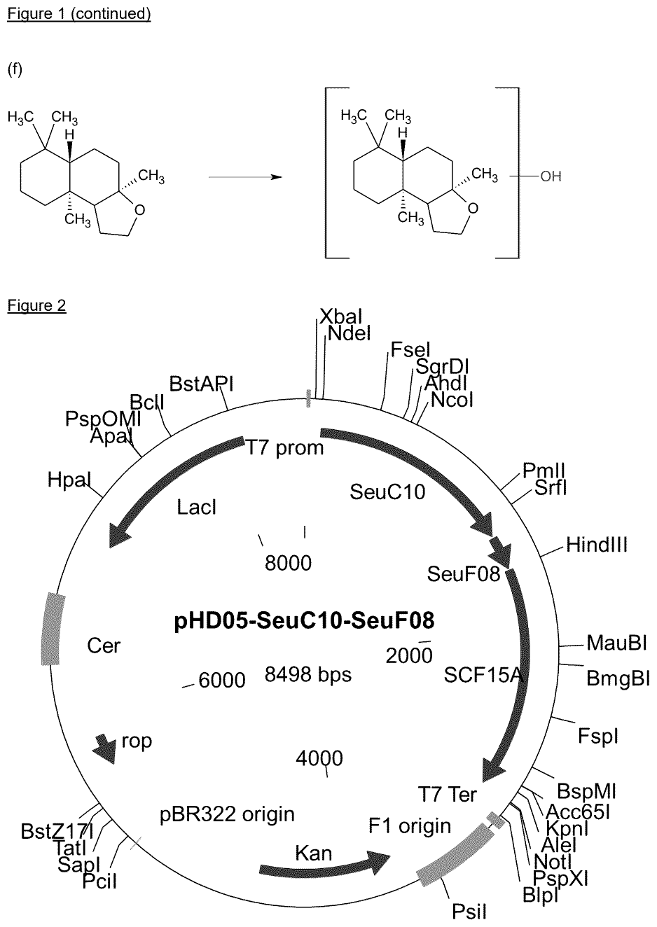

Measurement of the concentration of cytochrome P450 were performed according to the method of Omura and Sato et al. (J. Biol. Chem., 239. 1964, 2370). The cytochrome P450 concentration of cell-free extracts of induced E. coli Tuner (DE3) cells harbouring pHD05-SeuC10-SeuF08 was 17 μM. The carbon monoxide difference spectrum for P450_{SeuC10} is shown in FIG. 3.

### Example 4A: Oxidase Activity/Spectrum Testing

Lyophilised material of recombinant P450, ferredoxin and ferredoxin reductase proteins was made as described in Example 3 and reconstituted in high purity water to the original volume. Biocatalysis was performed shaking at 27° C. in the following conditions: 100 mM potassium phosphate pH 8.0, 5 mM MgCl₂ (both present in the reconstituted enzyme preparation) and 0.1 mg/ml substrate compound such as carbamazepine, bosentan, diclofenac, meloxicam, tivantinib or ambroxide. Concentrations of P450, ferredoxin and ferredoxin reductase were as extracted (Example 3). Reactions were initiated by addition of 10× stock of cofactor mixture (50 mM G6P, 10 mM NADP, 10 UN/ml G6PDH) to provide a final volume of e.g., 10 μL to 90 μL for 100 μL total reaction volumes. After 16-20 hours, reactions were extracted with an equal volume of acetonitrile, centrifuged to remove precipitated proteins and conversion assessed by UPLC-MS analysis.

UPLC data were obtained as follows:
Column: Acquity UPLC BEH Shield RP18 1.7 μm 2.1 mm i.d. 50 mm length
Solvents: H₂O, B: Acetonitrile, both with 0.1% Formic acid
Flow rate: 1.0 ml/min
Detector: Waters Acquity UPLC PDA (UV-Vis detection) and Waters Acquity UPLC QDA (MS)

To confirm the identities of reaction products where known, their chromatographic retention time, mass and ultraviolet spectra were compared with those of authentic metabolite standards.

Representative results for the overall % conversion to oxidised products are shown in Table 1 below.

TABLE 1
Results for substrate testing with lyophilised material
containing P450_{SeuC10} co-expressed with ferredoxin_{SeuF08}
and ferredoxin reductase_{SCF15A}.

| | Overall % Conversion to |
|---|---|
| Substrate | Oxidised Products |
| Ambroxide | 72.8 |
| Epothilone B | 35.1 |
| Diclofenac | 98.3 |
| Tivantinib | 86.9 |
| Meloxicam | 31.8 |
| Carbamazepine | 49.2 |
| Tolbutamide | 21 |
| Palmitic acid | 70.2 |

### Example 4B: Hydroxylase Activity/Spectrum Testing by Whole-Cell Biotransformation in E. coli

Cell pellets were provided as in Example 2. The cell pellets were defrosted, washed with 0.85% NaCl buffer and centrifuged at 4,000 RCF, 30 minutes at 4° C. The supernatant was discarded and the pellet was flash frozen in liquid nitrogen. The pellet was allowed to defrost again and resuspended in 40 mL of buffer containing 50 mM potassium phosphate, pH 7.4, 5 mM MgCl₂ and 100 mM glucose.

Biocatalysis was performed shaking at 27° C. with 0.1 mg/ml substrate compound such as carbamazepine, bosentan, diclofenac, meloxicam, tivantinib, ambroxide or others as shown in Table 2. Reactions were stopped and analysed as in Example 4. Results of wider substrate testing are shown in Table 2, below.

TABLE 2
Results of wider substrate testing for oxidation
by P450_{SeuC10} co-expressed in E. coli with
ferredoxin_{SeuF08} and ferredoxin reductase_{SCF15A}.

| | Overall % Conversion to |
|---|---|
| Substrate | Oxidised Products |
| Ritonavir | 21.6 |
| Buparvaquone | 23.4 |
| BIRB796 | 19.3 |
| Bosentan | 87.1 |
| Vanoxerine | 26.5 |
| Ruxolitinib | 38.6 |
| Perindopril | 14.4 |

### Example 5: Comparison of the Activity of P450_{SeuC10} with Other Cytochrome P450s

Other cytochrome P450 enzymes were expressed as described above, and their activities tested against the same substrates as P450_{SeuC10} using the methods described in Example 4. The other P450s tested were: P450_{AluC09} from Amycolatopsis lurida NRRL 2430 (SEQ ID NO: 13, see WO 2018/091885); SriC12 from Streptomyces rimosus NRRL 2234 (SEQ ID NO: 14, see WO 2020/109776); SriC20 from Streptomyces rimosus NRRL 2234 (SEQ ID NO: 15, see WO 2020/109776); SriC22 from Streptomyces rimosus NRRL 2234 (SEQ ID NO: 16, see WO 2020/109776); and CYP107L from Streptomyces platensis DSM 40041 (SEQ ID NO: 17, see Worsch et al., Biotechnology and Bioengineering 115:2156-2166, 2018).

The results are shown in Tables 3 and 4 below, which show the overall % percentage conversion to oxidised products of the substrates. The results reported in Tables 3 and 4, were generated using the methods described in Examples 4A and 4B, respectively, with the substrates as indicated in the tables below. The first column in each table corresponds to the results provided in Tables 1 and 2, above.

TABLE 3
Results for substrate testing with lyophilised
material containing the indicated P450

| Substrate | SeuC10 | AluC09 | SriC12 | SriC20 | SriC22 |
|---|---|---|---|---|---|
| Ambroxide | 72.8 | 0 | 0 | 0 | 0 |
| Epothilone B | 35.1 | 46 | 2 | 0 | 0 |
| Diclofenac | 98.3 | 89.9 | 33.9 | 0 | 0.9 |
| Tivantinib | 86.9 | 72 | 27.6 | 2.2 | 9.6 |
| Meloxicam | 31.8 | 25.1 | 73 | 0 | 0 |
| Carbamazepine | 49.2 | 0 | 3.2 | 0 | 0 |
| Tolbutamide | 21 | 0 | 34 | 0 | 0 |
| Palmitic acid | 70.2 | 28.5 | 35.5 | 0 | 0 |

TABLE 4
Results for substrate testing with whole-
cells containing the indicated P450

| Substrate | SeuC10 | AluC09 | CYP107L |
|---|---|---|---|
| Ritonavir | 21.6 | 86.3 | 47.5 |
| Buparvaquone | 23.4 | 48.8 | 8.8 |
| BIRB796 | 19.3 | 5.2 | 0 |
| Bosentan | 87.1 | 100 | 93 |
| Vanoxerine | 26.5 | 0 | 7.4 |
| Ruxolitinib | 38.6 | 34.3 | 16.3 |
| Perindopril | 14.4 | 0 | 0 |

As shown in the tables, the enzyme of the invention (SeuC10) demonstrates higher levels of conversions of almost all substrates than any other single cytochrome P450 tested.

1. A cytochrome P450 enzyme comprising the amino acid sequence set forth in SEQ ID NO: 3, or a variant thereof having an amino acid sequence having at least 95% identity thereto and having CYP450 activity.

2. The enzyme of claim 1, comprising a sequence having at least 96% identity to, more preferably 97% identity to, even more preferably 98% identity to, even more preferably 99% identity to, most preferably comprising, the amino acid sequence set forth in SEQ ID NO: 3.

3. A nucleic acid molecule comprising a nucleotide sequence encoding an enzyme as defined in claim 1 or 2.

4. A recombinant construct comprising the nucleic acid molecule of claim 3 operatively linked to a heterologous expression control sequence.

5. A vector comprising the nucleic acid molecule of claim 3 or the recombinant construct of claim 4.

6. A microorganism comprising the nucleic acid molecule of claim 3, the recombinant construct of claim 4 or the vector of claim 5, wherein the nucleotide sequence encoding the enzyme as defined in claim 1 or 2 is heterologous to the microorganism, or a lysate of said microorganism;
wherein preferably the microorganism is not Streptomyces eurythermus.

7. The microorganism of claim 6, wherein the microorganism is Escherichia coli.

8. Use of a cytochrome P450 enzyme as defined in claim 1 or 2, for the oxidation of an organic compound.

9. The use according to claim 8, wherein the oxidation is hydroxylation, epoxidation, carboxylation or dealkylation.

10. The use according to claim 8 or 9, wherein the organic compound to be oxidised comprises an alkyl, aryl or olefinic group, and/or an alkyl or aryl substituent.

11. The use according to any of claims 8 to 10, wherein the compound to be oxidised is of formula (I): 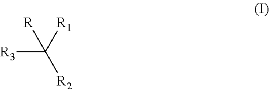
where R represents the rest of the compound, and where R¹, R² and R³ are independently selected from H or C₁₋₁₂ alkyl or C₆₋₁₀ aryl, or wherein any two of R¹, R² and R³ may be joined to form an optionally substituted cycloalkyl or heterocycloalkyl or R¹, R² and R³ may be joined together with their bridging carbon to form an olefin, aryl or heteroaryl.

12. The use according to any one of claims 8 to 11, wherein the compound to be oxidised is carbamazepine, bosentan, diclofenac, meloxicam, tivantinib or ambroxide.

13. The use according to any one of claims 8 to 12, wherein the cytochrome P450 enzyme is used in combination with reductase components or other reducing agents, preferably with ferredoxin and ferredoxin reductase components.

14. The use according to any one of claims 8 to 13, wherein the cytochrome P450 enzyme is in a purified form, a part-purified form, a crude enzyme extract, a recombinant host cell or a natural host cell.

15. The use according to claim 14, wherein the cytochrome P450 enzyme, or variant thereof is present in Streptomyces eurythermus NRRL 2539 cells.

16. The use according to any one of claims 8 to 15, wherein the cytochrome P450 enzyme, or variant thereof is expressed by at least one recombinant microorganism comprising a heterologous nucleic acid encoding the enzyme, preferably as defined in claim 6 or 7.

17. A method for the production of an oxidised organic compound, comprising reacting the organic compound with a cytochrome P450 enzyme as defined in claim 1 or 2.

18. The method according to claim 17, where the organic compound is oxidised by hydroxylation, epoxidation, carboxylation or dealkylation.

19. The method according to claim 17 or 18, wherein the organic compound to be oxidised is as defined in any one of claims 10 to 12.

20. The method according to any of claims 17 to 19, wherein the cytochrome P450 enzyme is used in combination with reductase components, preferably with ferredoxin and ferredoxin reductase components.

21. The method according to any of claims 17 to 20, wherein the cytochrome P450 enzyme is in a purified form, a part-purified form, a crude enzyme extract, a recombinant host cell or a natural host cell.

22. The method according to any of claims 17 to 21, wherein the cytochrome P450 enzyme, or variant thereof, is present in Streptomyces eurythermus NRRL 2539 cells and wherein said cells are dosed with an organic compound to be oxidised, optionally wherein the cells are subsequently harvested and the oxidised compound isolated.

23. The method according to any of claims 17 to 22, wherein the cytochrome P450 enzyme or variant thereof is expressed by at least one recombinant microorganism comprising a heterologous nucleic acid encoding the enzyme, preferably as defined in claim 6 or 7, wherein the at least one recombinant microorganism is dosed with an organic compound to be oxidised, optionally followed by a purification step to obtain the oxidised compound.

24. A kit comprising:
i) a cytochrome P450 enzyme as defined in claim 1 or 2;
ii) a microorganism that expresses a cytochrome P450 enzyme as defined in claim 1 or 2, preferably a microorganism as defined in claim 6 or 7, or a lysate thereof; and/or
iii) a nucleic acid molecule as defined in claim 3, a recombinant construct as defined in claim 4 or a vector as defined in claim 5.

25. The kit according to claim 24, wherein the kit further comprises a reducing agent, preferably a ferredoxin reductase and a ferredoxin, optionally wherein the kit further comprises a buffer.

26. The kit according to claim 24 or 25, further comprising one or more other cytochrome P450 enzyme and/or instructions for using the kit for the oxidation of an organic compound.

27. The kit according to any of claims 24 to 26, wherein the cytochrome P450 enzyme, microorganism or lysate, or nucleic acid molecule, recombinant construct or vector is lyophilised and/or vacuum sealed.

28. A method of producing a cytochrome P450 enzyme as defined in claim 1 or 2, the method comprising introducing a nucleic acid molecule as defined in claim 3, a recombinant construct as defined in claim 4 or a vector as defined in claim 5 into a microorganism, and expressing the cytochrome P450 enzyme in the microorganism, and optionally purifying the cytochrome P450 enzyme.
